(19) United States
(12) Patent Application Publication
(10) Pub. No.: US 2021/0188680 A1
(43) Pub. Date: Jun. 24, 2021

(54) WASTE TREATMENT DEVICE

(71) Applicants: SCG CHEMICALS CO., LTD, Bangkok (TH); INC SQUARE CO., LTD, Pathumthani (TH)

(72) Inventors: Attawut Kumkrong, Bangkok (TH); Atitaya Panuvatvanich, Pathumthani (TH); Thammarat Koottatep, Chiangmai (TH); Sompong Hirunmasuwan, Pathumthani (TH); Pantaporn Sittplangkoon, Pathumthani (TH); Araya Wicheansan, Pichit (TH); Sakkarin Meephon, Kalasin (TH)

(73) Assignees: SCG CHEMICALS CO., LTD, Bangkok (TH); INC SQUARE, CO., LTD, Pathumthani (TH)

(21) Appl. No.: 17/270,726

(22) PCT Filed: Aug. 22, 2019
(86) PCT No.: PCT/TH2019/000034

§ 371 Date: Feb. 23, 2021

(30) Foreign Application Priority Data
Aug. 24, 2018 (SG) 10201807199W

(51) Int. Cl. C02F 9/00; B01D 21/00; B01D 21/26; B01D 21/30; B01D 21/34; B09B 3/00; A61L 11/00; A61L 2/02; A61L 2/18; A61L 2/26

(52) U.S. Cl. CPC C02F 9/00; B01D 21/009; B01D 21/267; B01D 21/302; B01D 21/34; C02F 2001/007; A61L 11/00; A61L 2/022; A61L 2/18; A61L 2/26; B09B 3/0083

(57) ABSTRACT

A waste treatment device may include a solid-liquid separator which is configured to receive and separate waste into solids and liquid. The waste treatment device may further include a solids treatment arrangement which is configured to receive the solids from the solid-liquid separator, wherein the solids treatment arrangement comprises a disinfection unit having a heating mechanism configured to heat, without burning, the solids so as to disinfect the solids to convert the solids into pathogen-free-treated-solids. The waste treatment device may further include a liquid treatment arrangement which is configured to receive the liquid from the solid-liquid separator and to treat the liquid so as to convert the liquid into pathogen-free-effluent. The solid-liquid separator may include a curved-funnel-shaped inner separator surface and a frustoconically-shaped inner liquid guide surface whereby the respective narrower ends are directed towards each other.

### TECHNICAL FIELD

Various embodiments generally relate to a waste treatment device.

### BACKGROUND

Sewage or waste from a toilet or a lavatory, and/or waste from livestock farm, generally contains a mixture of solids (such as faeces) and liquid (such as urine and/or flush water). The solids from the sewage or waste generally contains total coliform bacteria such as faecal coliform, and the liquid from the sewage or waste generally contains contaminants. Accordingly, sewage or waste is not suitable for direct disposal or application in agriculture. This is because the coliform bacteria from the solids and the contaminants in the liquid can be harmful to the environment and public health if the sewage or waste is not treated to remove these harmful substances before being disposed or application in agriculture. Further, the high moisture content of the solids may also make it unsuitable for direct agriculture application. Generally, in urban places, sewage or waste would be treated in large sewage treatment facilities before being disposed and/or reused. However, in rural places, sewage or waste is generally left untreated and may pose a risk to the environment and public health if directly disposed and/or reused.

Accordingly, there is still a need for a waste treatment device that addresses at least some of the issues identified above.

### SUMMARY

According to various embodiments, there is provided a waste treatment device. The waste treatment device may include a solid-liquid separator which is configured to receive and separate waste into solids and liquid. The waste treatment device may further include a solids treatment arrangement which is configured to receive the solids from the solid-liquid separator, wherein the solids treatment arrangement comprises a disinfection unit having a heating mechanism configured to heat, without burning, the solids so as to disinfect the solids to convert the solids into pathogen-free-treated-solids. The waste treatment device may further include a liquid treatment arrangement which is configured to receive the liquid from the solid-liquid separator and to treat the liquid so as to convert the liquid into pathogen-free-effluent. The solid-liquid separator may include a curved-funnel-shaped inner separator surface configured to set the waste into spiral motion as the waste moves towards a spout of the curved-funnel-shaped inner separator surface and a frustoconically-shaped inner liquid guide surface. The spout of the curved-funnel-shaped inner separator surface and a narrower end of the frustoconically-shaped inner liquid guide surface may be directed towards each other.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments are described with reference to the following drawings, in which:

FIG. 1 shows a schematic diagram of a waste treatment device according to various embodiments;

FIG. 2A shows a schematic diagram of a waste treatment device according to various embodiments;

FIG. 2B shows a curvature of a curved-funnel-shaped inner separator surface of a solid-liquid separator of the waste treatment device of FIG. 2A according to various embodiments;

FIG. 3 shows a waste treatment device according to various embodiments;

FIG. 4 shows a casing structure of the waste treatment device of FIG. 3 without covers according to various embodiments;

FIG. 5 shows a casing structure of the waste treatment device of FIG. 3 without three removable covers and without a support frame according to various embodiments;

FIG. 6 shows the waste treatment device of FIG. 3 with part of the casing structure cut away to show the interior of the waste treatment device according to various embodiments;

FIG. 7 shows a top view of FIG. 6 according to various embodiments;

FIG. 8A and FIG. 8B shows a waste treatment device according to various embodiments; and

FIG. 9A and FIG. 9B shows a perspective view and a top view of the liquid treatment arrangement of the waste treatment device of FIG. 8 with a cover removed according to various embodiments.

### DETAILED DESCRIPTION

Embodiments described below in the context of the apparatus are analogously valid for the respective methods, and vice versa. Furthermore, it will be understood that the embodiments described below may be combined, for example, a part of one embodiment may be combined with a part of another embodiment.

It should be understood that the terms “on”, “over”, “top”, “bottom”, “down”, “side”, “back”, “left”, “right”, “front”, “lateral”, “side”, “up”, “down” etc., when used in the following description are used for convenience and to aid understanding of relative positions or directions, and not intended to limit the orientation of any device, or structure or any part of any device or structure. In addition, the singular terms “a”, “an”, and “the” include plural references unless context clearly indicates otherwise. Similarly, the word “or” is intended to include “and” unless the context clearly indicates otherwise.

Various embodiments generally relate to a waste treatment device. In particular, various embodiments relate to a waste treatment device for treating waste or sewage collected from a toilet or a lavatory or a livestock farm. Further, various embodiments relate to a single compact portable standalone waste treatment device that may be brought to rural places for direct treatment of waste or sewage collected in-situ before disposal or reusing the treated waste for agriculture. According to various embodiments, waste or sewage may refer to a mixture of solids and liquid from the waste or sewage collected.

Various embodiments seek to provide a waste treatment device that addresses the above-identified issues. Various embodiments seek to provide a waste treatment device that provides easy and fuss-free portable standalone all-in-one solution for direct treatment of waste or sewage in-situ in rural places without the need to fix up or install or connect multiple devices for processing the waste or sewage collected to separate the solids and liquid, and for separately treating the solids and liquid.

Various embodiments seek to provide a waste treatment device which is independent of city sewage treatment system. Various embodiments may be configured to be installed directly to independent toilet or lavatory found in rural areas for direct independent treatment of waste or sewage. Accordingly, various embodiments may be configured to be portable or easily transportable. Various embodiments seek to provide a compact portable all-in-one solution for separation of the solids and the liquid as well as treatment of both the solids and the liquid from the waste or sewage in one single device.

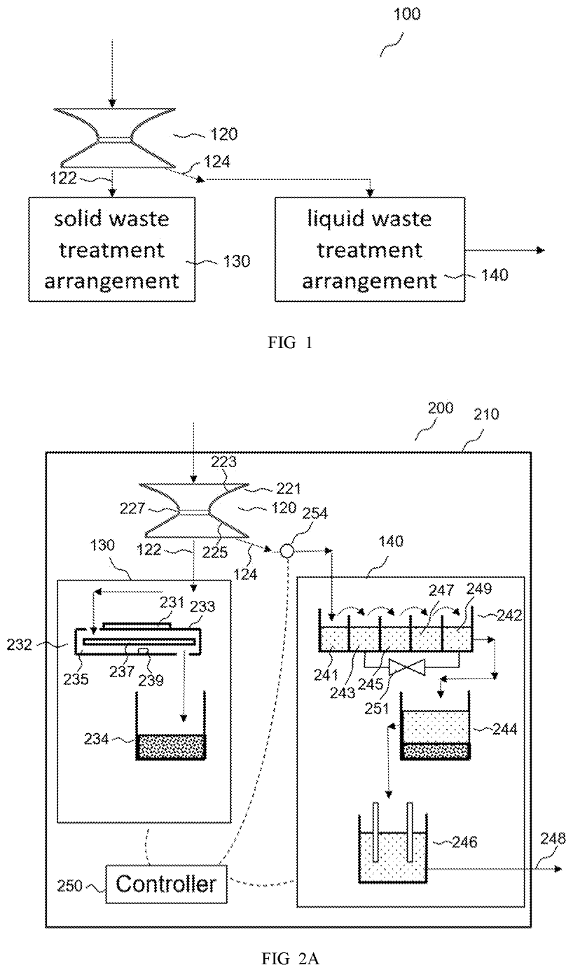

FIG. 1 shows a schematic diagram of a waste treatment device 100 according to various embodiments. As shown, the waste treatment device 100 may include a solid-liquid separator 120. According to various embodiments, the solid-liquid separator 120 may be configured to receive waste or sewage from the toilet or the lavatory or the livestock farm. Further, the solid-liquid separator 120 may be configured to separate waste into solids and liquid. Accordingly, waste or sewage from the toilet or the lavatory or the livestock farm may be supplied, or deposited, or fed into the waste treatment device 100 and may enter the solid-liquid separator 120. The solid-liquid separator 120 may separate the solids and liquid, and direct them to separate discharge outlets 122, 124.

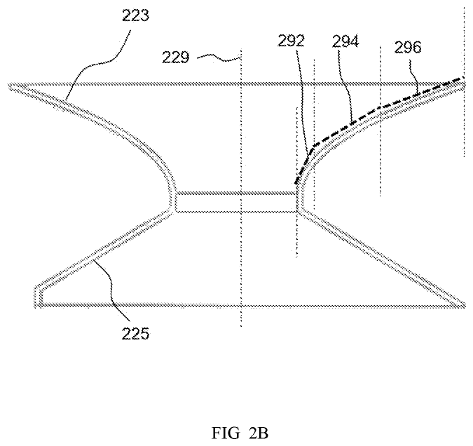

According to various embodiments, the waste treatment device 100 may include a solids treatment arrangement 130. The solids treatment arrangement 130 may be configured to receive the solids from a solid discharge outlet 122 of the solid-liquid separator 120. Further, the solids treatment arrangement 130 may be configured to disinfect the solids so as to convert the solids into pathogen-free-treated-solids. The solids treatment arrangement 130 may include a disinfection unit 232 (see FIG. 2A) having a heating mechanism 231 configured to heat, without burning, the solids so as to subject the solids to thermal disinfection to convert the solids into pathogen-free-treated-solids. The heating mechanism 231 of the disinfection unit 232 may also be configured to heat, without burning, the solids to remove moisture content from the solids so as to dry the solids. Accordingly, the solids treatment arrangement 130 may process or treat the solids such that pathogen, such as total coliform bacteria etc., may be removed from the solids. Hence, the solids treatment arrangement 130 may remove substances harmful to the environment and public health from the solids so that the pathogen-free-treated-solids may be safely disposed or reused. According to various embodiments, ‘heat, without burning,’ may refer to a state of a body having a high degree of warmth or a higher temperature, and exclude the state of the body being incinerated, cremated, combusted, ignited, set on fire, set ablaze, or reduced to ashes etc. Accordingly, the solids may be heated such that a temperature of the solids is raised, however, the solids are not in the state of being (or the solids are in a state free from being) burned, incinerated, cremated, combusted, ignited, set on fire, set ablaze, or reduced to ashes etc.

According to various embodiments, the waste treatment device 100 may include a liquid treatment arrangement 140. The liquid treatment arrangement 140 may be configured to receive the liquid from a liquid discharge outlet 124 of the solid-liquid separator 120. Further, the liquid treatment arrangement 140 may be configured to treat the liquid so as to convert the liquid into pathogen-free-effluent. Accordingly, the liquid treatment arrangement 140 may process or treat the liquid such that pathogen and contaminants, such as total coliform bacteria etc., may be removed from the liquid. Hence, the liquid treatment arrangement 140 may remove substances harmful to the environment and public health from the liquid so that the pathogen-free-effluent may be safely disposed or reused.

According to various embodiments, the solid-liquid separator 120, the solids treatment arrangement 130, and the liquid treatment arrangement 140 may be of separate modular construction such that the solid-liquid separator 120, the solids treatment arrangement 130, and the liquid treatment arrangement 140 may be contained within respective individual housing. Accordingly, each of the solid-liquid separator 120, the solids treatment arrangement 130, and the liquid treatment arrangement 140 may be a single module connectable with each other so as to form the waste treatment device 100. According to various embodiments, the solid-liquid separator 120, the solids treatment arrangement 130, and the liquid treatment arrangement 140 may be placed side-by-side to each other or in separate locations and may be joined or connected to each other via a piping arrangement. According to various embodiments, solid-liquid separator 120 may be integrated with either the solids treatment arrangement 130 or the liquid treatment arrangement 140 to form a single integral unit or module (i.e. contain within the same housing). Accordingly, the single module having the solid-liquid separator 120 and the solids treatment arrangement 130 (i.e. contain within the same housing) may be connected to a separate liquid treatment arrangement 140 external to the single module. On the other hand, single module having the solid-liquid separator 120 and the liquid treatment arrangement 140 (i.e. contain within the same housing) may be connected to a separate solids treatment arrangement 130 external to the single module. According to various embodiments, the solids treatment arrangement 130 and the liquid treatment arrangement 140 may be integrated to form a single integral unit or module (i.e. contain within the same housing). Accordingly, the single module having the solids treatment arrangement 130 and the liquid treatment arrangement 140 (i.e. contain within the same housing) may be connected to a separate solid-liquid separator 120 external to the single module. According to various embodiments, the waste treatment device 100 formed by interconnecting the various modules may provide an easy and simple plug-and-play solution for the separation of the waste or sewage into solids and the liquid as well as the treatment of both the solids and the liquid. According to various embodiments, the waste treatment device 100 formed by interconnecting the various modules may be contained within a single housing to form a single complete device.

FIG. 2A shows a schematic diagram of a waste treatment device 200 according to various embodiments. The waste treatment device 200 of FIG. 2A contains all the features of the waste treatment device 100 of FIG. 1. Accordingly, all features, changes, modifications, and variations that are applicable to the waste treatment device 100 of FIG. 1 are also applicable to the waste treatment device 200 of FIG. 2A. According to various embodiments, the waste treatment device 200 of FIG. 2A differs from the waste treatment device 100 of FIG. 1 in that the waste treatment device 200 of FIG. 2A may include the following additional features and/or limitations.

According to various embodiments, the solid-liquid separator 120, the solids treatment arrangement 130, and the liquid treatment arrangement 140 may be integrated into a single casing structure 210 as represented by a rectangular outline in FIG. 2A. It is understood that the rectangular outline in FIG. 2A is for illustration purposes only and does not limit the shape or the configuration of the single casing structure 210. The single casing structure 210 may be of any shapes and configurations. According to various embodiments, the single casing structure 210 may include two or more parts (or a plurality of parts) joined together to form a structural whole so as to provide an unitary exterior housing for the waste treatment device 100 such that the solid-liquid separator 120, the solids treatment arrangement 130, and the liquid treatment arrangement 140 may be incorporated into waste treatment device 100 and assembled or combined within or inside the single casing structure 210. Accordingly, the assemblage or combination of the single casing structure 210, the solid-liquid separator 120, the solids treatment arrangement 130, and the liquid treatment arrangement 140 may form a single complete whole device or an integral device which may provide a compact fuss-free portable standalone all-in-one solution for separation of the waste or sewage into solids and the liquid as well as treatment of both the solids and the liquid. According to various embodiments, the single casing structure 210 may be configured to be portable and of a compact size suitable to be transported or conveyed or carried to remote rural area by hand or other suitable manner.

According to various embodiments, the solid-liquid separator 120 may be configured to separate the waste or sewage into solids and liquid based on centrifugal force and gravity. The solid-liquid separator 120 may be configured to facilitate movement of the waste or sewage (i.e. a mixture of solids and liquid) in a manner whereby the solids and the liquid may be separated due to difference in momentum resulting in different movements between the solids and the liquid. The solid-liquid separator 120 may be configured to guide or direct or differentiate or demarcate different regions for collecting the solids and the liquid due to the resultant difference in movements of the solids and the liquid which cause the solids and the liquid to exit at the different regions of the solid-liquid separator 120. According to various embodiments, the solid-liquid separator 120 may be configured such that the waste or sewage may be set into a spiral motion as the waste or sewage enters the solid-liquid separator 120. Due to the difference in nature of the solids and the liquid, the gravity and the centrifugal force may influence the solids and the liquid differently such that the solids and the liquid may result in different momentum and spiral motion. Accordingly, the solids and liquid may move along different paths and may be separately collected from the solid-liquid separator 120 or separated by the solid-liquid separator 120. According to various embodiments, the solid-liquid separator 120 may be disposed within the single casing structure 210.

According to various embodiments, the solid-liquid separator 120 may include a hollow structure 221. The hollow structure 221 of the solid-liquid separator 120 may include a curved-funnel-shaped inner separator surface 223. The curved-funnel-shaped inner separator surface 223 may be an inner surface of the hollow structure 221 having a shape resembling a curved funnel, a vortex funnel, a curved cone, a trumpet shape (i.e. conical but with flaring at the broad end) or other similar shapes. According to various embodiments, the curved-funnel-shaped inner separator surface 223 may be configured to set the waste into spiral motion as the waste moves towards a spout of the curved-funnel-shaped separator surface. Accordingly, as the waste or the sewage moves along the curved-funnel-shaped separator surface, the gravity and the centrifugal force may influence the solids and the liquid differently so as to result in the difference in momentum of the solids and liquid such that the solids and the liquid move along different paths which may allow the solids and the liquid to be separately collected or separated. For example, the solids may move in a curve path towards the spout of the curved-funnel-shaped inner separator surface 223 and fall through the center of the spout of the curved-funnel-shaped inner separator surface 223. On the other hand, the liquid may flow or travel along the curved-funnel-shaped inner separator surface 223 in a spiral motion, resembling a vortex, while maintaining contact with the curved-funnel-shaped inner separator surface 223 as the liquid reaches the spout of the curved-funnel-shaped inner separator surface 223. According to various embodiments, the solids may fall through a centre of the spout of the curved-funnel-shaped separator surface while the liquid may flow along the curved-funnel-shaped separator surface to a rim of the spout of the curved-funnel-shaped separator surface.

According to various embodiments, a curvature of the curved-funnel-shaped inner separator surface 223 of the solid-liquid separator 120 may be defined by a smooth curve fitted to at least three straight lines arranged in a series. FIG. 2B shows the curvature of the curved-funnel-shaped inner separator surface 223 of the solid-liquid separator 120 of FIG. 2A according to various embodiments. According to various embodiments, respective angles of each successive straight line of the at least three straight lines with respect to the axis 229 of the curved-funnel-shaped inner separator surface 223 may be of increasing magnitude from an innermost straight line of the at least three straight lines to an outermost straight line of the at least three straight lines. Accordingly, an angle of the innermost straight line with respect to the axis 229 of the curved-funnel-shaped inner separator surface 223 may be smaller than an angle of an intermediate straight line with respect to the axis 229 of the curved-funnel-shaped inner separator surface 223, and the angle of the intermediate straight line with respect to the axis 229 of the curved-funnel-shaped inner separator surface 223 may be smaller than an angle of the outermost straight line with respect to the axis 229 of the curved-funnel-shaped inner separator surface 223.

As shown in FIG. 2B, the curvature of the curved-funnel-shaped inner separator surface 223 of the solid-liquid separator 120 may be defined by a smooth curve fitted to three straight lines 292, 294, 296 arranged in sequence one after another. Accordingly, the at least three straight lines may include the three successive straight lines 292, 294, 296 in a sequence of a first straight line 292 (or the innermost straight line) followed by a second straight line 294 (or the intermediate straight line) and followed by a third straight line 296 (or the outermost straight line). The first straight line 292 may form an angle between 20° to 35°, or between 25° to 30° with respect to the axis 229 of the curved-funnel-shaped inner separator surface 223. The second straight line 294 may form an angle between 50° to 65°, or between 55° to 60° with respect to the axis 229 of the curved-funnel-shaped inner separator surface 223. The third straight line 296 may form an angle between 65° to 75°, or between 67° to 72° with respect to the axis 229 of the curved-funnel-shaped inner separator surface 223. According to various embodiments, a lateral distance of the first straight line 292 may be approximately 15% of the entire lateral distance between the spout and the mouth of the curved-funnel-shaped inner separator surface 223. According to various embodiments, a lateral distance of the second straight line 294 may be approximately 40% of the entire lateral distance between the spout and the mouth of the curved-funnel-shaped inner separator surface 223. According to various embodiments, a lateral distance of the third straight line 296 may be approximately 45% of the entire lateral distance between the spout and the mouth of the curved-funnel-shaped inner separator surface 223. According to various embodiments, the curvature of the curved-funnel-shaped inner separator surface 223 as defined above may effectively separate the solids and the liquid as the solid-liquid mixture move along the curved-funnel-shaped inner separator surface 223 from the mouth of the curved-funnel-shaped inner separator surface 223 to the spout of the curved-funnel-shaped inner separator surface 223.

According to various embodiments, the solid-liquid separator 120 may include a frustoconically-shaped inner liquid guide surface 225. Referring back to FIG. 2A, the frustoconically-shaped inner liquid guide surface 225 may be an inner surface of the hollow structure 221 having a shape resembling a frustum of a cone, a truncated cone, or a tapering circular sidewall. According to various embodiments, the spout of the curved-funnel-shaped inner separator surface 223 and a narrower end of the frustoconically-shaped inner liquid guide surface 225 may be directed towards each other. According to various embodiments, the frustoconically-shaped inner liquid guide surface 225 may receive the liquid flowing or travelling from the spout of the curved-funnel-shaped inner separator surface 223 and further guide the liquid to flow or travel along the frustoconically-shaped inner liquid guide surface 225 from the narrower end to a broader end of the frustoconically-shaped inner liquid guide surface 225. Accordingly, liquid may continue to flow onto the frustoconically-shaped inner liquid guide surface 225 upon exiting the spout of the curved-funnel-shaped separator surface. On the other hand, solids falling though the centre of the spout of the curved-funnel-shaped separator surface may continue to fall along a centre axis of the frustoconically-shaped inner liquid guide surface 225. Thus, liquid may be collected at a periphery of the frustoconically-shaped inner liquid guide surface 225, while the solids may be collected along the centre axis of the frustoconically-shaped inner liquid guide surface 225.

According to various embodiments, a ratio of a difference in radius between the broader end and the narrower end of the frustoconically-shaped inner liquid guide surface 225 to a height of the frustoconically-shaped inner liquid guide surface 225 may be between 1.2 to 2.75, or between 1.43 to 2.14. Accordingly, the frustoconically-shaped inner liquid guide surface 225 may form an angle of between 50° to 70°, or between 55° to 65° with respect to the axis of the frustoconically-shaped inner liquid guide surface 225. According to various embodiments, the slant of the frustoconically-shaped inner liquid guide surface 225 as defined above may effectively guide most of the liquid along the frustoconically-shaped inner liquid guide surface 225 from the narrower end of the frustoconically-shaped inner liquid guide surface 225 to the broader end of the frustoconically-shaped inner liquid guide surface 225.

According to various embodiments, the solid-liquid separator 120 further comprises a conduit portion 227. The spout of the curved-funnel-shaped inner separator surface 223 may be directly connected to a first end of the conduit portion 227 and the narrower end of the frustoconically-shaped inner liquid guide surface 225 may be directly connected to a second end of the conduit portion 227. Accordingly, the conduit portion 227 may be disposed between the curved-funnel-shaped inner separator surface 223 and the frustoconically-shaped inner liquid guide surface 225 such that the conduit portion 130 forms a connection or linkage between the curved-funnel-shaped inner separator surface 223 and the frustoconically-shaped inner liquid guide surface 225 for fluid communication. Hence, the solids and the liquid may pass through the conduit portion from the curved-funnel-shaped inner separator surface 223 to the frustoconically-shaped inner liquid guide surface 225. Accordingly, the solids which is separated from the liquid as a result of the waste or sewage being set to motion on the curved-funnel-shaped inner separator surface 223 and which fall through the center of the spout of the curved-funnel-shaped inner separator surface 223 may continue its falling motion through the center of the conduit portion 223 and through the center of the frustoconically-shaped inner liquid guide surface 225. On the other hand, the liquid which is separated from the solids as a result of the waste or sewage being set to motion on the curved-funnel-shaped inner separator surface 223 and which flows or travels along the curved-funnel-shaped inner separator surface 223 in a spiral motion to the spout of the curved-funnel-shaped inner separator surface 223 may continue to flow or travel along the inner surface of the conduit portion 227 (i.e. continue to remain in contact with the inner surface of the conduit portion 227) to the narrower end of the frustoconically-shaped inner liquid guide surface 225 and may then continue to flow or travel along the frustoconically-shaped inner liquid guide surface 225 towards the broader end of the frustoconically-shaped inner liquid guide surface 225 (i.e. continue to remain in contact with the frustoconically-shaped inner liquid guide surface 225). Thus, according to various embodiments, the solids may be collected from a centre of the frustoconically-shaped inner liquid guide surface 225 and the liquid may be collected from the periphery of the frustoconically-shaped inner liquid guide surface 225.

According to various embodiments, a ratio of a length of the conduit portion 227 to a height of the curved-funnel-shaped inner separator surface 223 may be equal or less than 0.2, or between 0.1 to 0.2. According to various embodiments, a short length of the conduit portion 227 may minimise the re-mixing of the solids with the liquid while both the solids and the liquid are passing through the conduit portion 227. The short length of the conduit portion 227 may also allow the liquid flowing or travelling from the spout of the curved-funnel-shaped inner separator surface 223 to cross over to the frustoconically-shaped inner liquid guide surface 225 upon exiting the conduit portion 227 while maintaining contact with the respective inner surfaces.

According to various embodiments, which are not shown, various embodiments may also have negligible conduit portion or may not even include a conduit portion. Accordingly, the curved-funnel-shaped inner separator surface 223 of the solid-liquid separator 120 may be directly connected to the frustoconically-shaped inner liquid guide surface 225 of the solid-liquid separator 120.

According to various embodiments, the curved-funnel-shaped inner separator surface 223 and/or the frustoconically-shaped inner liquid guide surface 225 and/or the inner surface of the conduit portion 227 of the solid-liquid separator 120 may be coated with hydrophobic material. According to various embodiments, the curved-funnel-shaped inner separator surface 223 and/or the frustoconically-shaped inner liquid guide surface 225 and/or the inner surface of the conduit portion 227 may be made of hydrophobic material. According to various embodiments, the solid-liquid separator 120 according to the various embodiments may be made of hydrophobic material. According to various embodiment, due to the hydrophobicity of the respective inner surfaces as described above, liquid may form droplets (with a contact angle between 150° to 170° with respect to the respective inner surfaces) as the liquid flows or travels along the respective inner surfaces such that the liquid may remain in contact with the respective inner surfaces as the liquid flows or travels.

As shown in FIG. 2A, the solids treatment arrangement 130 may include the disinfection unit 232. According to various embodiments, the disinfection unit 232 may be disposed within the single casing structure 210. Further, the disinfection unit 232 may be configured to apply thermal disinfection and/or drying to the solids. According to various embodiments, the solids separated by the solid-liquid separator 120 may be supplied or fed or deposited into the disinfection unit 232 of the solids treatment arrangement 130. A solid discharge outlet 122 of the solid-liquid separator 120 may be connected to an inlet of the disinfection unit 232 of the solids treatment arrangement 130. Accordingly, solids may be supplied or fed or deposited into the disinfection unit 232 of the solids treatment arrangement 130 via the connection between the solid discharge outlet 122 of the solid-liquid separator 120 and the inlet of the disinfection unit 232 of the solids treatment arrangement 130. According to various embodiments, the disinfection unit 232 may be configured to subject the solids received from the solid-liquid separator 120 to a predetermined disinfection temperature. Accordingly, the disinfection unit 232 may be configured to heat, without burning, the solids to disinfect the solids so as to provide the pathogen-free-treated-solids. According to various embodiments, the predetermined disinfection temperature may be a temperature that is sufficient to kill the pathogen and may not be a temperature that would burn or incinerate the solids. Thus, the disinfection unit 232 may be configured to thermal disinfect the solids without burning or incinerating or cremating or combusting or igniting or setting ablaze the solids, or setting the solids on fire, or reducing the solids to ashes. Accordingly, the disinfection unit 232 of the solids treatment arrangement 130 may convert the solids into the pathogen-free-treated-solids. According to various embodiments, the disinfection unit 232 may also be configured to heat, without burning, the solids to remove moisture content from the solids so as to dry the solids. Accordingly, the predetermined disinfection temperature may be a temperature that is sufficient to kill the pathogen and to dry the solids without burning the solids. Hence, the pathogen-free-treated-solids may also be dried by the disinfection unit 232 during the thermal disinfection process. According to various embodiments, the predetermined disinfection temperature may be at least 70° C., or between 70° C. to 200° C., or between 90° C. to 200°, or between 100° C. to 200°, or between 70° C. to 150° C., or between 90° C. to 150°, or between 100° C. to 150°, or between 70° C. to 130° C., or between 90° C. to 130°, or between 100° C. to 130° or between 115° C. to 125° C., or about 120° C. For example, a temperature above 70° C. may already be suitable for thermal disinfection. Further, a temperature above 100° C. may be suitable for both thermal disinfection and drying.

According to various embodiments, the heating mechanism 231 of the disinfection unit 232 may be configured to heat an elongate housing 233 of the disinfection unit 232 so as to heat an internal space 235 of the elongate housing 233 to create a heated environment to apply thermal disinfection and/or drying to the solids received inside the elongate housing 233 from the solid-liquid separator 120. Accordingly, the heating mechanism 231 may transfer heat to the elongate housing 233 such that the heated elongate housing 233 may heat the internal space 235 of the elongate housing 233 to provide the heated environment for thermal disinfection and/or drying. Hence, the internal space 235 of the elongate housing 233 may be heated to the predetermined disinfection temperature to provide the heated environment for thermal disinfection and/or drying. Thus, the solids which is deposited into the elongate housing 233 of the disinfection unit 232 may be exposed to the heated environment inside the elongate housing 233, without being burned or incinerate or cremated or combusted or ignited or set on fire or set ablaze or reduced to ashes, for thermal disinfection and/or drying of the solids. According to various embodiments, the elongate housing 233 may include an inlet and an outlet.

According to various embodiments, the heating mechanism 231 may be configured to apply direct contact heating to the elongate housing 233. According to various embodiments, the heating mechanism 231 may be configured to wrap around or surround the elongate housing 233 such that heating mechanism 231 may provide a uniform heating around the elongate housing 233 or circumferentially. According to various embodiments, the heating mechanism 231 may include one or more heating elements (or a plurality of heating elements) lined in sequence along the length of the elongate housing 233 to provide a uniform heating along the length of the elongate housing 233 or lengthwise. Accordingly, the heating mechanism 231 may include one or more heating elements directly coupled to the elongate housing 233. According to various embodiments, the heating mechanism may include a band heater, or a heater pad, or a heater plate, or a heating net, or a heater coil, or a heater wire, or a heater rod, or a heater fin, or any combination thereof. According to various embodiments, the one or more heating mechanism, preferably, include the band heater.

According to various embodiments, the disinfection unit 232 may include a conveying mechanism 237 configured to move the solids along and within the elongate housing 233 of the disinfection unit 232. The conveying mechanism 237 may extend at least substantially along a length of the elongate housing 233 from the inlet of the elongate housing 233 to the outlet of the elongate housing 233. Accordingly, the conveying mechanism 237 may be contained inside the elongate housing 233 and may be disposed such that the extent of the conveying mechanism 237 stretches from the inlet of the housing 233 to the outlet of the housing 233. Hence, the solids which enters the inlet of the housing 233 may be conveyed or transported or carried or moved or transferred by the conveying mechanism 237 along the elongate housing 233 and/or to the outlet of the elongate housing 233. According to various embodiments, the conveying mechanism 237 may include a screw conveyor mechanism, or a bucket conveyor mechanism, or a drag chain conveyor mechanism, or a belt conveyor mechanism, or a wire mesh conveyor mechanism, or a roller conveyor mechanism, or a spiral conveyor mechanism, or any other suitable conveyor mechanism that may convey or transport or carry or move or transfer solids from a first longitudinal end portion of the elongate housing 233 to a second opposite longitudinal end portion of the housing 233. According to various embodiments, the conveying mechanism 237 may, preferably, include the screw conveyor mechanism.

According to various embodiments, the disinfection unit 232 may further include one or more temperature sensors 239 disposed and configured to measure a temperature of the internal space 235 of the elongate housing 233. Accordingly, the one or more temperature sensors 239 may provide feedback regarding a temperature of the heated environment within the elongate housing 233. According to various embodiments, the one or more temperature sensors 239 may be disposed at or within the elongate housing 233. According to various embodiments, the one or more temperature sensors 239 may be located or disposed at any point or position at or within the housing 233. According to various embodiments, the one or more temperature sensors 239 may include thermocouple, or resistance temperature detector, or semiconductor-based sensor, or temperature detector with multiple sensing points, or other suitable type of temperature sensing devices.

According to various embodiments, the solids treatment arrangement 130 may further include a collector unit 234. The collector unit 234 may be disposed within the single casing structure 210. Further, the collector unit 234 may be configured to receive the pathogen-free-treated-solids from the disinfection unit 232. According to various embodiments, an outlet of the elongate housing of the disinfection unit 232 may be connected to an inlet of the collector unit 234. Accordingly, pathogen-free-treated-solids (which may also be dried) may be supplied or fed or deposited into the collector unit 234 of the solids treatment arrangement 130 via the connection between the outlet of the elongate housing of the disinfection unit 232 and the inlet of the collector unit 234. The collector unit 234 may function as a storage for the pathogen-free-treated-solids such that pathogen-free-treated-solids may be accumulated to a certain amount before it is disposed or re-used.

As shown in FIG. 2A, the liquid treatment arrangement 140 may include a biological treatment unit 242. According to various embodiments, the biological treatment unit 242 may be disposed within the single casing structure 210. According to various embodiments, the biological treatment unit 242 may be configured to remove organic substances in the liquid.

Accordingly, the biological treatment unit 242 may remove or reduce organic contaminants in the liquid. According to various embodiments, the liquid separated by the solid-liquid separator 120 may be supplied or fed or flow into the biological treatment unit 242 of the liquid treatment arrangement 140. A liquid discharge outlet 124 of the solid-liquid separator 120 may be in fluid communication with an inlet of the biological treatment unit 242 of the liquid treatment arrangement 140. Accordingly, the liquid may be supplied or fed or flow into the biological treatment unit 242 of the liquid treatment arrangement 140 via the fluid communication between the liquid discharge outlet 124 of the solid-liquid separator 120 and the inlet of the biological treatment unit 242 of the liquid treatment arrangement 140.

According to various embodiments, the biological treatment unit 242 of the liquid treatment arrangement 140 may include a filtration chamber 241, or an anaerobic treatment chamber 243, or an aerobic treatment chamber 245, or an anoxic treatment chamber 247, or any combination thereof. According to various embodiments, the filtration chamber 241 may be configured to receive the liquid from the solid-liquid separator 120. The filtration chamber 241 may be configured to filter away solid particles that may have accidentally exit from the liquid outlet 124 of the solid-liquid separator 120. According to various embodiments, the filtration chamber 241 may include a plurality of plastic media. According to various embodiments, each of the plurality of plastic media may be of about 5 cm in size. According to various embodiments, the anaerobic treatment chamber 243 may be configured to expose the liquid to anaerobic bacteria (in a no oxygen condition) to remove organic matter from the liquid. According to various embodiments, the anaerobic treatment chamber 243 may include a plurality of plastic media and spherical clay media. According to various embodiments, each of the plurality of plastic media may be of about 5 cm in size and each of the spherical clay media may be of about 2 cm in diameter According to various embodiments, the aerobic treatment chamber 245 may be configured to expose the liquid to bacteria that require oxygen to remove organic matter. According to various embodiments, the aerobic treatment chamber 245 may include a plurality of spherical clay media and an aeration mechanism to supply air bubbling in the aerobic treatment chamber 245. The air bubbling may help to remove the organic substances in the liquid. According to various embodiments, each of the plurality of spherical clay media may be of about 1 cm in diameter. According to various embodiments, the aeration mechanism may be an air pump. According to various embodiments, the anoxic treatment chamber 247 may be configured to remove nitrogen from liquid (in a no oxygen condition) via biological nitrogen removal process (or denitrification). According to various embodiments, the anoxic treatment chamber 247 may include a plurality of zeolite. According to various embodiments, each of the plurality of zeolite may be between 0.3 cm to 2 cm in size. According to various embodiments, the biological treatment unit 242 may include the filtration chamber 241, the anaerobic treatment chamber 243, the aerobic treatment chamber 245, and the anoxic treatment chamber 247. Further, the biological treatment unit 242 may be configured to flow the liquid through the respective chambers in a sequence of the filtration chamber 241 followed by the anaerobic treatment chamber 243, followed by the aerobic treatment chamber 245, and followed by the anoxic treatment chamber 247. Accordingly, the biological treatment unit 242 may be arranged with the filtration chamber 241, the anaerobic treatment chamber 243, the aerobic treatment chamber 245, and the anoxic treatment chamber 247 in a series such that the liquid may flow through the filtration chamber 241 into the anaerobic treatment chamber 243, through the anaerobic treatment chamber 243 into the aerobic treatment chamber 245, and through the aerobic treatment chamber 245 into the anoxic treatment chamber 247. According to various embodiments, the biological treatment unit 242 may include a holding chamber 249 after the anoxic treatment chamber 247. Accordingly, the liquid may flow through the anoxic treatment chamber 247 into the holding chamber 249.

According to various embodiments, the biological treatment unit 242 may include a recirculation pump 251 configured to recirculate the liquid through the respective chambers 243, 245, 247. According to various embodiments, the recirculation pump 251 may be in fluid communication with the holding chamber 249 and the anaerobic treatment chamber 243. Accordingly, the recirculation pump 251 may be configured to draw some of the liquid from the holding chamber 249 and pump into the anaerobic treatment chamber 243 such that the liquid may be recirculated through the anaerobic chamber 243, the aerobic treatment chamber 245, and the anoxic treatment chamber 247.

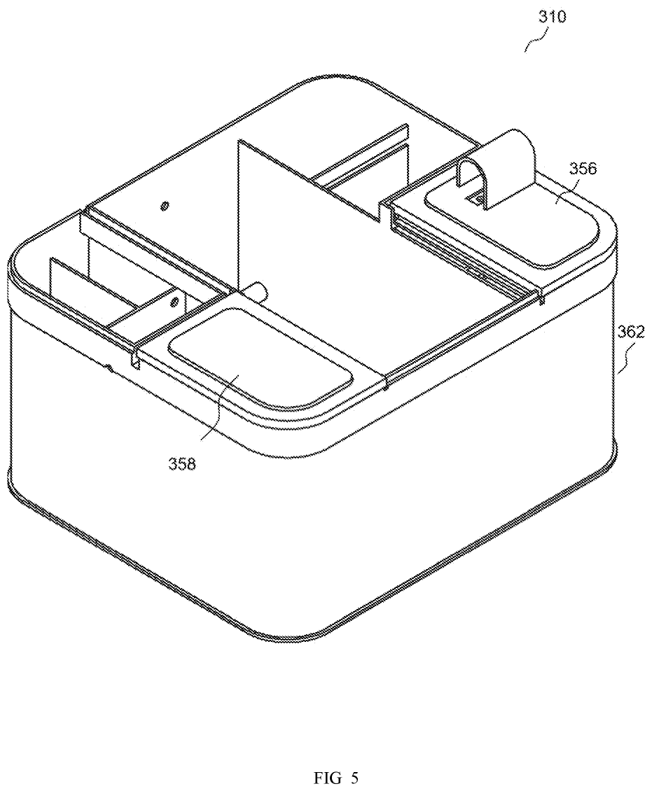

According to various embodiments, the respective chambers of the biological treatment unit 242 of the liquid treatment arrangement 140 may be internal spaces partitioned within the single casing structure 210 of the waste treatment device 200. Accordingly, the single casing structure 210 of the waste treatment device 200 may include a plurality of partition walls 370 (see FIG. 4) to divide the internal spaces of the single casing structure 210 into the respective chambers of the biological treatment unit 242 of the liquid treatment arrangement 140.

According to various embodiments, the liquid treatment arrangement 140 may include a sedimentation unit 244. The sedimentation unit 244 may be is disposed within the single casing structure 210. Further, the sedimentation unit 244 may be configured to remove suspension particle in the liquid. According to various embodiments, the sedimentation unit 244 may be configured to remove suspended particle via gravity whereby the suspended particles are allowed to settle at the bottom of the sedimentation unit 244. According to various embodiments, the liquid treated by biological treatment unit 242 of the liquid treatment arrangement 140 may be supplied or fed or flow into the sedimentation unit 244 of the liquid treatment arrangement 140. An outlet of the biological treatment unit 242 of the liquid treatment arrangement 140 may be in fluid communication with an inlet of the sedimentation unit 244 of the liquid treatment arrangement 140. For example, an outlet of the holding chamber 249 of the biological treatment unit 242 of the liquid treatment arrangement 140 may be in fluid communication with an inlet of the sedimentation unit 244 of the liquid treatment arrangement 140. Accordingly, the liquid treated by the biological treatment unit 242 of the liquid treatment arrangement 140 may be supplied or fed or flow into the sedimentation unit 244 of the liquid treatment arrangement 140 via the fluid communication between the outlet of the biological treatment unit 242 of the liquid treatment arrangement 140 and the inlet of the sedimentation unit 244 of the liquid treatment arrangement 140.

According to various embodiments, the liquid treatment arrangement 140 may include an electrochemical unit 246. The electrochemical unit 246 may be disposed within the single casing structure 210. Further, the electrochemical unit 246 may be configured to oxidize chloride-ion in the liquid to chlorine for disinfection of the liquid. Accordingly, direct electrolysis may be applied to the liquid, which may have passed through the biological treatment unit 242 and the sedimentation unit 244, so as to oxidize chloride dissolved in the liquid to free chlorine for disinfection of the liquid. According to various embodiments, the liquid from the sedimentation unit 244 of the liquid treatment arrangement 140 may be supplied or fed or flow into the electrochemical unit 246 of the liquid treatment arrangement 140. An outlet of the sedimentation unit 244 of the liquid treatment arrangement 140 may be in fluid communication with an inlet of the electrochemical unit 246 of the liquid treatment arrangement 140. Accordingly, the liquid from sedimentation unit 244 of the liquid treatment arrangement 140 may be supplied or fed or flow into the electrochemical unit 246 of the liquid treatment arrangement 140 via the fluid communication between the outlet of the sedimentation unit 244 of the liquid treatment arrangement 140 and the inlet of the electrochemical unit 246 of the liquid treatment arrangement 140.

According to various embodiments, the sedimentation unit 244 and the electrochemical unit 246 of the liquid treatment arrangement 140 may be separate chambers formed within the single casing structure 210 of the waste treatment device 200. Accordingly, the sedimentation unit 244 and the electrochemical unit 246 of the liquid treatment arrangement 140 may be internal spaces partitioned within the single casing structure 210 of the waste treatment device 200. Hence, the single casing structure 210 of the waste treatment device 200 may include a plurality of partition walls 370 to divide the internal spaces of the single casing structure 210 into the sedimentation unit 244 and the electrochemical unit 246.

According to various embodiments, the liquid treatment arrangement 140 may include the biological treatment unit 242, or the sedimentation unit 244, or the electrochemical unit 246, or any combination thereof. According to various embodiments, the liquid treatment arrangement 140 may include the biological treatment unit 242, the sedimentation unit 244, and the electrochemical unit 246. Accordingly, the liquid treatment arrangement 140 may be configured to flow the liquid through the respective units in a sequence of the biological treatment unit 242 followed by the sedimentation unit 244 and followed by the electrochemical unit 246. Hence, the liquid treatment arrangement 140 may be arranged with the biological treatment unit 242, the sedimentation unit 244, and the electrochemical unit 246 in a series along a fluid communication line such that the liquid may flow through the biological treatment unit 242 into the sedimentation unit 244, and through the sedimentation unit 244 into the electrochemical unit 246. Thus, the liquid may be treated by the liquid treatment arrangement 140 to convert the liquid into pathogen-free-effluent.

According to various embodiments, the liquid treatment arrangement 140 may further include an effluent outlet 248 configured to discharge the pathogen-free-effluent out of the single casing structure 210 of the waste treatment device 200. Accordingly, the effluent outlet 248 may be in fluid communication with the electrochemical unit 246 such that pathogen-free-effluent obtained after the final treatment may be discharged through the effluent outlet 248.

According to various embodiments, the waste treatment device 200 may include a controller 250. The controller 250 may be understood as any kind of a logic implementing entity, which may be special purpose circuitry or a processor executing software stored in a memory, firmware, or any combination thereof. Thus, the controller 250 may be a hard-wired logic circuit or a programmable logic circuit such as a programmable processor (e.g. Programmable Logic Controller (PLC)), e.g. a microprocessor (e.g. a Complex Instruction Set Computer (CISC) processor or a Reduced Instruction Set Computer (RISC) processor). The controller may also be a processor executing software, e.g. any kind of computer program, e.g. a computer program using a virtual machine code such as e.g. Java. According to various embodiments, the controller 250 may be integrated in the device of the various embodiments or may be a separate device connected to the device of the various embodiments.

According to various embodiments, the waste treatment device 200 may also include a fluid sensing sensor 254 (or a fluid sensing switch) disposed any point from the liquid discharge outlet 124 of the solid-liquid separator 120 to the liquid treatment arrangement 140. For example, the fluid sensing sensor 254 may be disposed at the liquid discharge outlet 124 of the solid-liquid separator 120 or along the fluid communication between the solid-liquid separator 120 and the liquid treatment arrangement 140. According to various embodiments, the fluid sensing sensor 254 may be configured to detect presence of liquid. Accordingly, the fluid sensing sensor 254 may detect the liquid separated from the solid-liquid separator 120 as the liquid is being discharged from the liquid discharge outlet 124 of the solid-liquid separator 120 into the liquid treatment arrangement 140 each time the waste (or a mixture of solids and liquid) is passed through the solid-liquid separator 120. Hence, the fluid sensing sensor 254 may be configured as a measure to detect or indicate or register each time waste is passed through the solid-liquid separator 120. According to various embodiments, when the waste treatment device 200 is installed to a toilet or a lavatory, the waste from the toilet or the lavatory may pass through the solid-liquid separator 120 whenever the toilet or the lavatory is flushed. Thus, the fluid sensing sensor 254 may be configured as a measure to detect or indicate or register each flush of the toilet or the lavatory. According to various embodiments, the fluid sensing sensor 254 may include a normally opened circuit with two contact points disposed at the liquid discharge outlet 124 of the solid-liquid separator 120 or along the fluid communication between the solid-liquid separator 120 and the liquid treatment arrangement 140. Accordingly, when liquid passes through the liquid discharge out 124 of the solid-liquid separator 120 to the liquid treatment arrangement 140, the liquid may spread across the two contact points to close the circuit. Thus the normally opened circuit, which may be closed by the liquid stretching across the two contact points, may detect the presence of the liquid. According to various other examples, the fluid sensing sensor 254 may include optical sensor or capacitive sensor or float switch or resistance (or impedance) detection sensor or any other suitable sensors.

According to various embodiments, the controller 250 may be electrically coupled to the one or more temperature sensors 239 of the disinfection unit 232 of the solid treatment arrangement 130, or the heating mechanism 231 of the disinfection unit 232 of the solid treatment arrangement 130, or the conveying mechanism 237 of the disinfection unit 232 of the solid treatment arrangement, or the electrochemical unit 246 of the liquid treatment arrangement 130, or the fluid sensing sensor 254, or any combination thereof.

As shown in FIG. 2A, according to various embodiments, the controller 250 may be electrically coupled to the one or more temperature sensors 239 of the disinfection unit 232 of the solid treatment arrangement 130, the heating mechanism 231 of the disinfection unit 232 of the solid treatment arrangement 130, the conveying mechanism 237 of the disinfection unit 232 of the solid treatment arrangement, the electrochemical unit 246 of the liquid treatment arrangement 130, and the fluid sensing sensor 254.

Accordingly, the controller 250 may be configured to receive signals from the fluid sensing sensor 254 regarding the detection of the flow of liquid from the solid-liquid separator 120 to the liquid treatment arrangement 140. The controller 250 may also be configured to receive signals from the one or more temperature sensors 139 of the disinfection unit 232 of the solid treatment arrangement 130 regarding the temperature detected in the internal space 235 of the elongate housing 233 of the disinfection unit 232 of the solid treatment arrangement 130. Further, the controller 250 may be configured to send instructions to the electrochemical unit 246 of the liquid treatment arrangement 130, the heating mechanism 231 of the disinfection unit 232 of the solid treatment arrangement 130 and the conveying mechanism 237 of the disinfection unit 232 of the solid treatment arrangement 130 to operate, or activate, or control, or command the electrochemical unit 246, the heating mechanism 231 and the conveying mechanism 237 respectively.

According to various embodiments, the controller 250 may be configured to control the electrochemical unit 246 based on feedback from the fluid sensing sensor 254. Accordingly, the controller 250 may be configured to activate or operate the electrochemical unit 246 to start the electrolysis process upon detection of liquid flow from the solid-liquid separator 120 to the liquid treatment arrangement 140 by the fluid sensing sensor 254. Hence, the electrochemical unit 246 may be activated or operated each time the waste is passed through the solid-liquid separator 120. Thus, when the waste treatment device 200 is installed to a toilet or a lavatory, the electrochemical unit 246 may be activated or operated each time the toilet or the lavatory is flushed since waste may pass through the solid-liquid separator 120 each time the toilet or the lavatory is flushed. According to various embodiments, the controller 250 may be configured to activate or operate the electrochemical unit 246 for a predetermined duration before the electrochemical unit 246 is turned off or put on standby or put on power saving mode etc. Accordingly, the electrochemical unit 246 may be turned off or put on standby or put on power saving mode etc. after the predetermined duration each time electrochemical unit 246 is activated or operated.

According to various embodiments, the controller 250 may be configured to control the conveying mechanism 237 of the disinfection unit 232 of the solid treatment arrangement 130 based on feedback from the fluid sensing sensor 254. According to various embodiments, the controller 250 may be configured to count the number of times the fluid sensing sensor 254 detects liquid flow from the solid-liquid separator 120 to the liquid treatment arrangement 140 as a measure of the number of times waste is passed through the solid-liquid separator 120. According to various embodiments, the controller 250 may be configured to activate or operate the conveying mechanism 237 of the disinfection unit 232 of the solid treatment arrangement 130 based on a predetermined number of times the fluid sensing sensor 254 detects flow of liquid from the solid-liquid separator 120 to the liquid treatment arrangement 140. Accordingly, the conveying mechanism 237 of the disinfection unit 232 of the solid treatment arrangement 130 may be configured to be activated or operated by the controller 250 after the predetermined number of times waste is passed through the solid-liquid separator 120. According to various embodiments, when the waste treatment device 200 is installed to a toilet or a lavatory, the conveying mechanism 237 of the disinfection unit 232 of the solid treatment arrangement 130 may be configured to be activated or operated by the controller 250 after the predetermined number of times the toilet or the lavatory is flushed since waste may pass through the solid-liquid separator 120 each time the toilet or the lavatory is flushed. According to various embodiments, the predetermined number of times may be between 10 times to 20 times. According to various embodiments, when the conveying mechanism 237 is activated or operated after the predetermined number of times, the conveying mechanism 237 may be configured to move the solids inside the elongate housing 233 of the disinfection unit 232 of the solid treatment arrangement 130 for a predetermined distance along the elongate housing 233 so as to free up a space in the inlet region of the elongate housing 233 of the disinfection unit 232 of the solid treatment arrangement 130 such that more solids may be accumulated in the housing 233 of the disinfection unit 232 of the solid treatment arrangement 130 from subsequent waste input to the waste treatment device 200.

According to various embodiments, the controller 250 may be configured to activate or operate the heating mechanism 231 of the disinfection unit 232 of the solid treatment arrangement 130 based on a pre-set timing. According to various embodiments, the pre-set timing may be 12 midnight, or lam, or 2 am, etc. Accordingly, the heating mechanism 231 of the disinfection unit 232 of the solid treatment arrangement 130 may be activated or operated at a certain pre-specified time of each day.

According to various embodiments, the controller 250 may be configured to control the heating mechanism 231 of the disinfection unit 232 of the solid treatment arrangement 130 based on feedback from the one or more temperature sensors 239 of the disinfection unit 232 of the solid treatment arrangement 130 to control the temperature of the internal space 235 of the elongate housing 233 of the disinfection unit 232 of the solid treatment arrangement 130 for thermal disinfection and/or drying of the solids. Hence, the controller 250 may be configured to control the heating mechanism 231 of the disinfection unit 232 of the solid treatment arrangement 130 based on feedback from the one or more temperature sensors 239 of the disinfection unit 232 of the solid treatment arrangement 130 to maintain the heated environment within the elongate housing 233 of the disinfection unit 232 of the solid treatment arrangement 130 at the predetermined disinfection temperature. Accordingly, the controller 250, the one or more temperature sensors 239 of the disinfection unit 232 of the solid treatment arrangement 130, and the heating mechanism 231 of the disinfection unit 232 of the solid treatment arrangement 130 may form a closed-loop temperature control system to manage the temperature of the internal space 235 within the elongate housing 233 of the disinfection unit 232 of the solid treatment arrangement 130. Hence, the temperature of the internal space 235 within the elongate housing 233 of the disinfection unit 232 of the solid treatment arrangement 130 may be maintained or regulated such that the heated environment is at a constant predetermined disinfection temperature. Thus, the controller 250 may be configured to control the heating mechanism 231 of the disinfection unit 232 of the solid treatment arrangement 130 to maintain the temperature of the internal space 235 of the elongate housing 233 of the disinfection unit 232 of the solid treatment arrangement 130 at the predetermined disinfection temperature so as to maintain the heated environment within the elongate housing 233 of the disinfection unit 232 of the solid treatment arrangement 130.

According to various embodiments, when the heated environment with the predetermined disinfection temperature is formed within the internal space 235 of the elongate housing 233 of the disinfection unit 232 of the solid treatment arrangement 130, the controller 250 may be configured to control the conveying mechanism 237 of the disinfection unit 232 of the solid treatment arrangement 130 to repeatedly move in a first operation direction (or first direction) and in a second reverse direction (or second direction) so as to move the solids in a first longitudinal direction and in a second opposite longitudinal direction along the elongate housing of the disinfection unit 232 of the solid treatment arrangement 130 based on a predetermined sequence. According to various embodiments, the first longitudinal direction and the second opposite longitudinal direction may be along the length of the elongate housing 233 of the disinfection unit 232 of the solid treatment arrangement 130. Accordingly, the conveying mechanism 237 of the disinfection unit 232 of the solid treatment arrangement 130 may be controlled to move the solids up and down along the length of the elongate housing 233 of the disinfection unit 232 of the solid treatment arrangement 130 based on the predetermined sequence. According to various embodiments, when the conveying mechanism 237 of the disinfection unit 232 of the solid treatment arrangement 130 is a screw conveyor mechanism, the first operation direction may be a clockwise direction of the screw of the conveying mechanism 237 and the second reverse direction may be a counter-clockwise direction of the screw of the conveying mechanism 237. Accordingly, the screw of the screw conveyor mechanism 237 of the disinfection unit 232 of the solid treatment arrangement 130 may be controlled to rotate clockwise and counter-clockwise based on the predetermined sequence so as to move the waste up (or in the first longitudinal direction) and down (or in the second longitudinal direction) along the length of the elongate housing 233 of the disinfection unit 232 of the solid treatment arrangement 130. According to various embodiments, the predetermined sequence may include an order of movement of the conveying mechanism 237 in the respective directions and time allocated to each movement. According to various embodiments, an equal amount of time may be allocated to each movement. According to various embodiments, the sequence of movement of the conveying mechanism 237 to move the waste up and down along the length of the elongate housing 233 may repeatedly mix and stir the solids in order to uniformly heat the solids to enhance the thermal disinfection and/or drying process of the solids.

According to various embodiments, the predetermined sequence may be performed or conducted within a predetermined period of time. Accordingly, the predetermined sequence may be performed or conducted by the controller during the predetermined period of time. According to various embodiments, at the end of the predetermined sequence and/or at the end of the predetermined period of time, the controller may be configured to control the conveying mechanism 237 of the disinfection unit 232 of the solid treatment arrangement 130 to convey or transport or carry or move or transfer the solids along the elongate housing 233 to the outlet of the elongate housing 233 such that the solids may exit the elongate housing 233 through the outlet. According to various embodiments, the predetermined period of time may be approximately between 60 minutes to 120 minutes (1 hour to 2 hours), or about 120 minutes (2 hours). According to various embodiments, the controller 250 may be configured to control the heating mechanism 231 of the disinfection unit 232 of the solid treatment arrangement 130 to heat the internal space 235 within the housing 233 of the disinfection unit 232 of the solid treatment arrangement 130 so as to maintain or regulate the temperature of the internal space 235 within the housing 233 at the predetermined disinfection temperature of the heated environment while the conveying mechanism 237 of the disinfection unit 232 of the solid treatment arrangement 130 is in operation.

According to various other embodiments, when the heated environment with the predetermined disinfection temperature is formed within the internal space 235 of the elongate housing 233 of the disinfection unit 232 of the solid treatment arrangement 130, the controller 250 may be configured to activate the conveying mechanism 237 of the disinfection unit 232 of the solid treatment arrangement 130 only after the internal space 235 of the elongate housing 233 of the disinfection unit 232 of the solid treatment arrangement 130 is maintained at the predetermined disinfection temperature for the predetermined period of time. Accordingly, the solids are accumulated in the elongate housing 233 of the disinfection unit 232 of the solid treatment arrangement 130 may be kept or maintained or retained inside the elongate housing 233 of the disinfection unit 232 of the solid treatment arrangement 130 for the predetermined period of time in the heated environment to undergo thermal disinfection and/or drying, before the conveying mechanism 237 of the disinfection unit 232 of the solid treatment arrangement 130 is activated by the controller 250 to convey or transport or carry or move or transfer the solids along the elongate housing 233 or to the outlet of the elongate housing 233 such that the solids may exit the elongate housing 233 of the disinfection unit 232 of the solid treatment arrangement 130 through the outlet. According to various embodiments, the predetermined period of time may be approximately between 60 minutes to 120 minutes (1 hour to 2 hours), or about 120 minutes (2 hours).

According to various embodiments, the controller 250 may be configured to maintain the internal space 235 of the elongate housing 233 of the disinfection unit 232 of the solid treatment arrangement 130 at the predetermined disinfection temperature (i.e. maintain the heated environment within the elongate housing 233) while the conveying mechanism 237 of the disinfection unit 232 of the solid treatment arrangement 130 is in operation. Accordingly, after the conveying mechanism 237 of the disinfection unit 232 of the solid treatment arrangement 130 is activated by the controller 250 to convey or transport or carry or move or transfer the waste, the controller 250 may continue to control the temperature of the internal space 235 inside the elongate housing 233 of the disinfection unit 232 of the solid treatment arrangement 130 via controlling the heating mechanism 231 of the disinfection unit 232 of the solid treatment arrangement 130 based on feedback from the one or more temperature sensors 239 of the disinfection unit 232 of the solid treatment arrangement 130 so as to maintain the internal space 235 of the elongate housing 233 of the disinfection unit 232 of the solid treatment arrangement 130 at the predetermined disinfection temperature to maintain the heated environment for further thermal disinfection and/or drying of the solids as the solids is in motion.

According to various embodiments, the controller 250 may be configured to control the heating mechanism 231 of the disinfection unit 232 of the solid treatment arrangement 130 and the conveying mechanism 237 of the disinfection unit 232 of the solid treatment arrangement 130 so as to disinfect and/or dry the solids deposited, supplied or fed into the elongate housing 233 of the disinfection unit 232 of the solid treatment arrangement 130. Accordingly, the controller 250 may be configured to control the heating mechanism 231 of the disinfection unit 232 of the solid treatment arrangement 130 and the conveying mechanism 237 of the disinfection unit 232 of the solid treatment arrangement 130 to remove pathogen, such as total coliform bacteria etc., from the solids. Further, the controller 250 may be configured to control the heating mechanism 231 of the disinfection unit 232 of the solid treatment arrangement 130 and the conveying mechanism 237 of the disinfection unit 232 of the solid treatment arrangement 130 to reduce moisture content of the waste from 84% to 99% of total mass to 41% to 52% of total mass (as measured based on the standard test methods for moisture—ASTM D 2974-87).

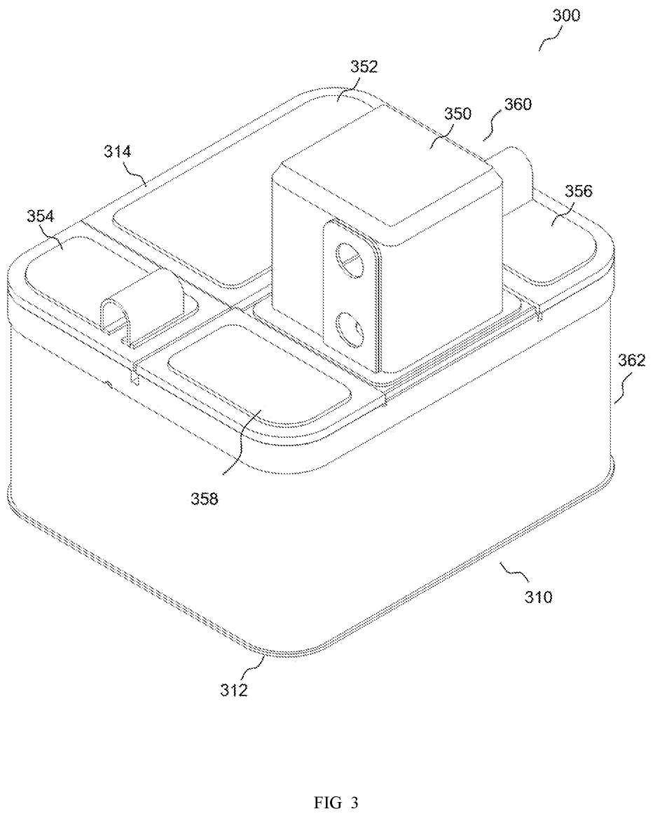

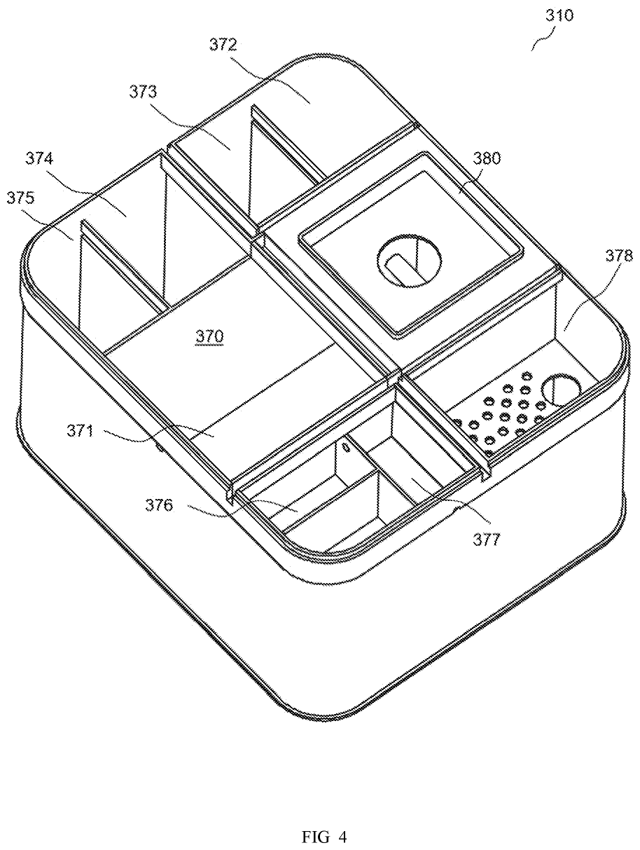

FIG. 3 shows a waste treatment device 300 according to various embodiments. The waste treatment device 300 of FIG. 3 contains all the features of the waste treatment device 100 of FIG. 1 and the waste treatment device 200 of FIG. 2A. Accordingly, all features, changes, modifications, and variations that are applicable to the waste treatment device 100 of FIG. 1 and the waste treatment device 200 of FIG. 2A are also applicable to the waste treatment device 300 of FIG. 3. According to various embodiments, the waste treatment device 300 of FIG. 3 differs from the waste treatment device 100 of FIG. 1 and the waste treatment device 200 of FIG. 2A in that the waste treatment device 300 of FIG. 3 may include the following additional features and/or limitations.

As shown in FIG. 3, the waste treatment device 300 may be a single complete whole device or an integral device which may be a compact fuss-free portable standalone all-in-one device for waste treatment. In FIG. 3, only the exterior of the single casing structure 310 may be seen. The solid-liquid separator 120, the solids treatment arrangement 130, and the liquid treatment arrangement 140 which are integrated into the single casing structure 310 and which are contained within the single casing structure 310 are not visible from FIG. 3.

As shown in FIG. 3, the single casing structure 310 may include a base part 312 and a cover part 314 which may be joined together to form a single structural whole. Accordingly, the base part 312 and the cover part 314 may be joined together to form a single complete casing for the waste treatment device 300. According to various embodiments, the base part 312 may be a one-piece container structure and the cover part 314 may include one or more cover members. Accordingly, the base part 312 may include an opening in the one-piece container structure which may be covered over or concealed by the one or more cover members of the cover part 314. According to various embodiments, the one or more cover members of the cover part 314 may be of any shape and sizes, and may be pieced together along respective boundaries to form the cover part 314 for covering or concealing the opening of the base part 312. As shown in FIG. 3, according to various embodiments, the cover part 314 may include five cover members 350, 352, 354, 356, 358. Four of the cover members may be flat or panel-like lids 352, 354, 356, 358. One of the cover members may be a protruding hollow cover 350 (or a shell-like enclosing type cover) which may enclose an additional space above the base part 312 to extend the internal space of the single casing structure 310 beyond the space confined by the base part 312 of the single casing structure 310.

According to various embodiments, the single casing structure 310 may include an upper level 360 and a lower level 362. The upper level 360 may be configured to define a space for the solid-liquid separator 120 such that the solid-liquid separator 120 may be housed in the upper level 360. Further, the lower level 362 may be configured to define a separate space for the solids treatment arrangement 130 and the liquid treatment arrangement 140 such that the solids treatment arrangement 130 and the liquid treatment arrangement 140 may be co-located in the lower level 362. As shown in FIG. 3, the upper level 360 may be defined by the protruding hollow cover 350 which encloses the additional space above the base part 312 while the lower level 362 may be defined by the space confined by the base part 312.

According to various embodiments, the lower level 362 may include at least one partitioning wall 370 (see FIG. 4) to divide the lower level into two or more chambers for demarcating separate areas (or separate portions of the space) for the solids treatment arrangement 130 and the liquid treatment arrangement 140 within the lower level 362. Accordingly, at least one chamber may be assigned or allocated to the solids treatment arrangement 130 and at least one other chamber may be assigned or allocated to the liquid treatment arrangement 140.

According to various embodiments, the single casing structure 310 may be partitioned into three or more internal spaces for separately locating the solid-liquid separator 120, the solids treatment arrangement 130 and the liquid treatment arrangement 140 within the single casing structure 310. Accordingly, the solid-liquid separator 120, the solids treatment arrangement 130 and the liquid treatment arrangement 140 may be assigned or allocated to different or separate portions (or areas) of the internal space within the single casing structure 310. According to various embodiments, the different or separate portions (or areas) of the internal space within the single casing structure 310 may be demarcated or marked by physical barriers or boundaries.

FIG. 4 shows the casing structure 310 of the waste treatment device 300 of FIG. 3 without covers according to various embodiments. As shown, the lower level 362 may be partitioned by partitioning walls 370 into eight chambers 371, 372, 373, 374, 375, 376, 377, 378. The lower level 362 may be partitioned to have a middle chamber 371 configured to define a space for accommodating the solids treatment arrangement 130. Further, the lower level 362 may be partitioned to have four side chambers 372, 373, 374, 375 on a first side of the middle chamber 371 and another three side chambers 376, 377, 378 on opposite second side of the middle chamber 371, whereby the first side and the second side are on opposite sides of the middle chamber 371. Accordingly, the side chambers 372, 373, 374, 375, 376, 377, 378 may be assigned or allocated to the liquid treatment arrangement 140. For example, the four side chambers 372, 373, 374, 375 on the first side of the middle chamber 371 may be assigned or allocated for the various chambers (e.g. the anaerobic treatment chamber 243, the aerobic treatment chamber 245, the anoxic treatment chamber 247, and the holding chamber 249) of the biological treatment unit 242 of the liquid treatment arrangement 140. Further, a first side chamber 376 of the three side chambers on the second side of the middle chamber 371 may be assigned or allocated for the sedimentation unit 244 of the liquid treatment arrangement 140. A second side chamber 377 of the three side chambers on the second side of the middle chamber 371 may be assigned or allocated for the electrochemical unit 246. A third side chamber 378 of the three side chambers on the second side of the middle chamber 371 may be the filtration chamber 251 of the biological treatment unit 242 for receiving and filtering the liquid from the solid-liquid separator 120 before supplying or feeding or flowing the liquid into the remaining chambers of the biological treatment unit 242.

According to various embodiments, the single casing structure 310 may include a suspended support frame 380. The suspended support frame 380 may separate the lower level 362 and the upper lever 360. Further, the suspended support frame 380 may be configured to support the solid-liquid separator 120. Accordingly, the suspended support frame 380 may be configured to be fitted to the base part 312 of the single casing structure 310 so as to be suspended at a portion of the opening of the base part 312 and may be configured to have sufficient rigidity to support the solid-liquid separator 120 which may be placed or fitted on the suspended support frame 380. According to various embodiments, the suspended support frame 380 may be overhanging from a side wall of the base part 312 of the single casing structure 310. Accordingly, the suspended support frame 380 may extend perpendicularly from the side wall of the base part 312 of the single casing structure 310.

According to various embodiments, the single casing structure 310 may include one or more removable covers. The one or more removable covers may be configured to be easily removed by a user of the waste treatment device 300 for accessing the solid-liquid separator 120, the solids treatment arrangement 130, and the liquid treatment arrangement 140 within the single casing structure 310 for maintenance or repair or removal of pathogen-free-treated-solids. Out of the five cover members 350, 352, 354, 356, 358 of the cover part 314 of the single casing structure 310, three cover members 350, 352, 354 may be made removable. FIG. 5 shows the single casing structure 310 of the waste treatment device 300 of FIG. 3 without the three removable cover members 350, 352, 354 according to various embodiments. As shown in FIG. 5, the three removable cover members 350, 352, 354 have been removed and only two cover members 356, 358 remains. Accordingly, the cover member 350 of the single casing structure 310 may be a removable cover for enclosing the solid-liquid separator 120. The single casing structure 310 may also include two other removable cover members 352, 354 (or at least one other removable cover) to cover over a portion of the lower level 362 of the single casing structure 310 for enclosing a portion of the solids treatment arrangement 130 and a portion of the liquid treatment arrangement 140. Further, in FIG. 5, it is also shown that the suspended support frame 380 may be made removable such that the solid-liquid separator 120 may be removed for maintenance or repair.

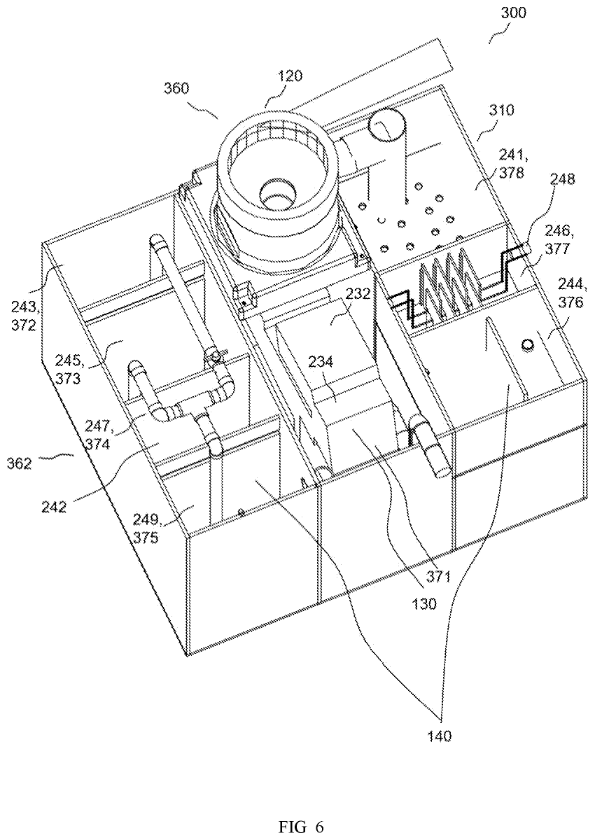

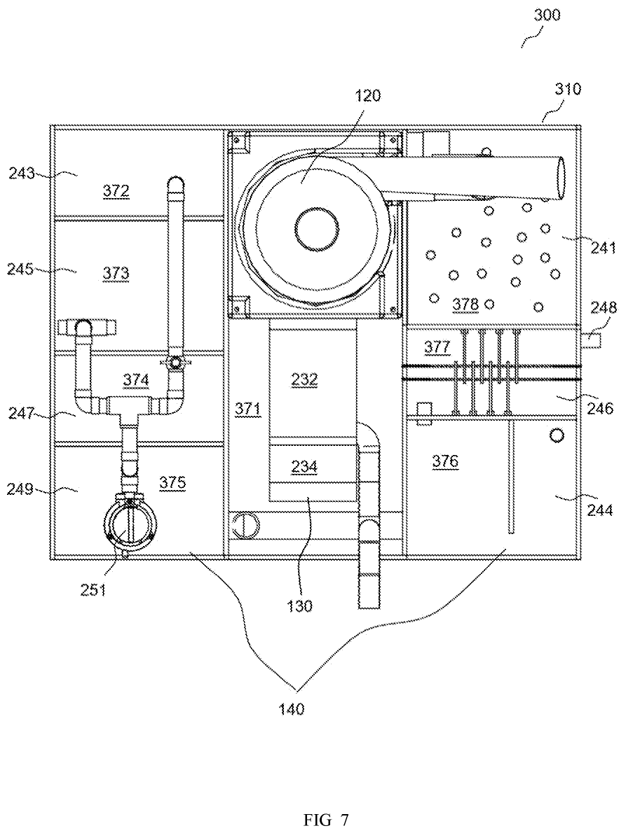

FIG. 6 shows the waste treatment device 300 of FIG. 3 with part of the single casing structure 310 cut away to show the interior of the waste treatment device 300 according to various embodiments. FIG. 7 shows a top view of FIG. 6 according to various embodiments. As shown, the solid-liquid separator 120, the solids treatment arrangement 130 and the liquid treatment arrangement 140 may be integrated into the single casing structure 310. Accordingly, the disinfection unit 232 and the collector unit 234 of the solids treatment arrangement 130 may be disposed or located within the single casing structure 310. The biological treatment unit 242, the sedimentation unit 244, and the electrochemical unit 246 may also be disposed or located within the single casing structure 310. As shown, the solid-liquid separator 120 may be assigned or allocated to the upper level 360 of the single casing structure 310. The disinfection unit 232 and the collector unit 234 of the solids treatment arrangement 130 may be assigned or allocated to the middle chamber 371 of the lower level 362 (or the base part 312) of the single casing structure 310. The biological treatment unit 242 (with the anaerobic treatment chamber 243, the aerobic treatment chamber 245, the anoxic treatment chamber 247, and the holding chamber 249) of the liquid treatment arrangement 140 may be assigned or allocated to the four side chambers 372, 373, 374, 375 on the first side of the middle chamber 371 of the lower level 362 (or the base part 312) of the single casing structure 310. The filtration chamber 241 of the biological treatment unit 242 may be assigned or allocated to the first side chamber 378 on the second side of the middle chamber 371 of the lower level 362 (or the base part 312) of the single casing structure 310. The electrochemical unit 246 may be assigned or allocated to the second side chamber 377 of the three side chambers on the second side of the middle chamber 371 of the lower level 362 (or the base part 312) of the single casing structure 310. The sedimentation unit 244 of the liquid treatment arrangement 140 may be assigned or allocated to the third side chamber 376 of the three side chambers on the second side of the middle chamber 371 of the lower level 362 (or the base part 312) of the single casing structure 310.

While an arrangement or an assignment or an allocation of space of the solid-liquid separator 120, the solids treatment arrangement 130 and the liquid treatment arrangement 140 within the single casing structure 310 of the waste treatment device 300, and/or the partitioning of the single casing structure 310 are shown in FIG. 3 to FIG. 7, it is understood that FIG. 3 to FIG. 7 are provided for illustration purposes and various embodiments may include changes, modification, variation in the arrangement or the assignment or the allocation of space of the solid-liquid separator 120, the solids treatment arrangement 130 and the liquid treatment arrangement 140 within the single casing structure 310 of the waste treatment device 300, and/or the partitioning of the single casing structure 310.

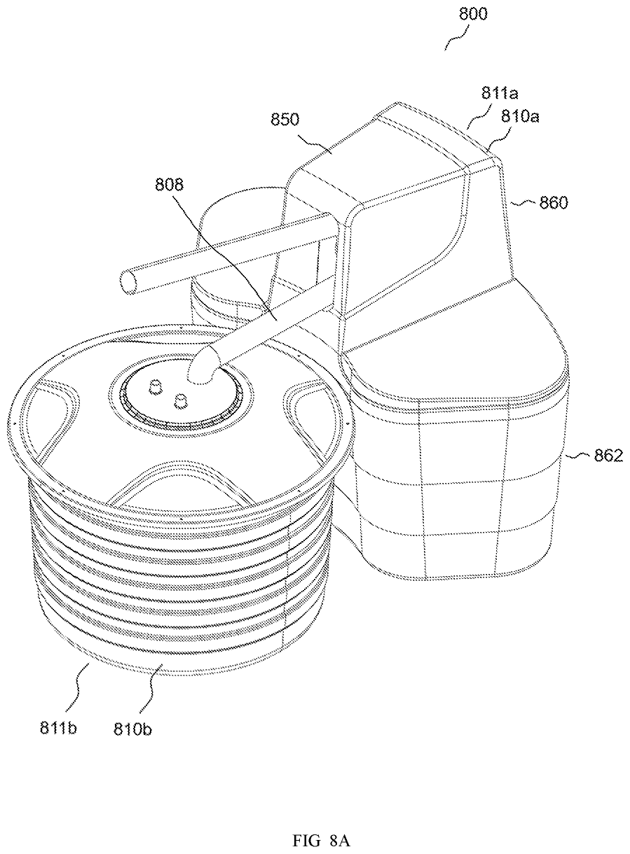

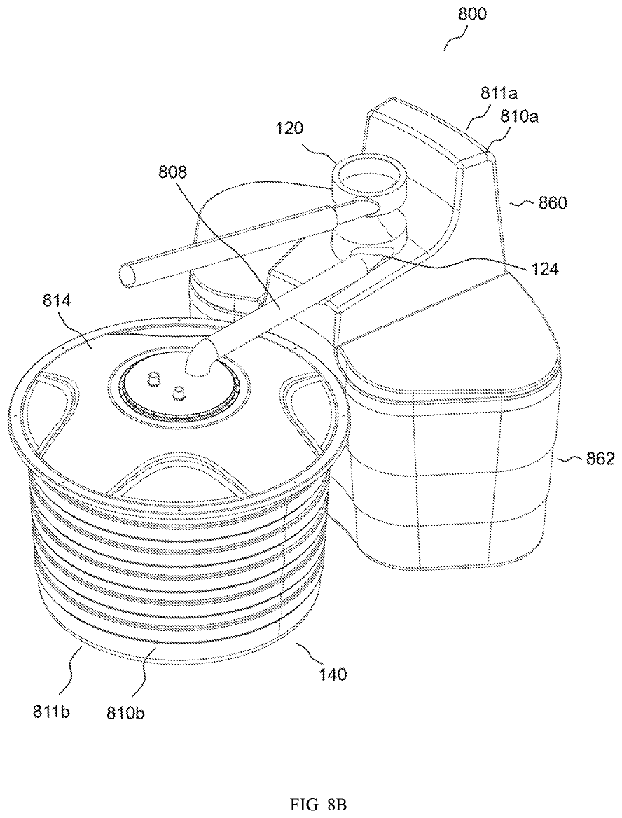

FIG. 8A and FIG. 8B shows a waste treatment device 800 according to various embodiments. The waste treatment device 800 of FIG. 8A and FIG. 8B contains all the features of the waste treatment device 100 of FIG. 1. Accordingly, all features, changes, modifications, and variations that are applicable to the waste treatment device 100 of FIG. 1 are also applicable to the waste treatment device 800 of FIG. 8A and FIG. 8B. According to various embodiments, the waste treatment device 800 of FIG. 8A and FIG. 8B differs, mainly, from the waste treatment device 200 of FIG. 2A and the waste treatment device 300 of FIG. 3 in that the waste treatment device 800 of FIG. 8A and FIG. 8B has the solid-liquid separator 120 and the solids treatment arrangement 130 integrated to form a single integral unit or module (i.e. contain within the same housing) and the liquid treatment arrangement 140 forms a separate unit or module (i.e. having its own separate housing) in a manner such that the single integral unit or module having the solid-liquid separator 120 and the solids treatment arrangement 130 (i.e. contain within the same housing) may be connected externally to the separate liquid treatment arrangement 140 (i.e. having its own separate housing), for example, via piping arrangements. Accordingly, other than features relating to the above main difference, the waste treatment device 800 of FIG. 8A and FIG. 8B may contain the remaining features of the waste treatment device 200 of FIG. 2A and the waste treatment device 300 of FIG. 3. In view of the above main difference, the waste treatment device 800 of FIG. 8A and FIG. 8B may include the following additional features and/or limitations.

As shown in FIG. 8A, the waste treatment device 800 may include two separate units 811a, 811b (or modules) which may be interconnected via a piping 808. According to various embodiments, the waste treatment device 800 formed by interconnecting the two separate units 811a, 811b may provide an easy and simple compact fuss-free portable plug-and-play solution for waste treatment. In FIG. 8A, only the exterior of casing structure 810a, 810b of the respective units 811a, 811b may be seen. The solid-liquid separator 120 and the solids treatment arrangement 130 may be integrated into the first casing structure 810a to form the first unit 811a. The liquid treatment arrangement 140 may be embodied by the second casing structure 810b to form the second unit 811b.

According to various embodiments, the first casing structure 810a of the first unit 811a may include an upper level 860 and a lower level 862. The upper level 860 may be configured to define a space for the solid-liquid separator 120 such that the solid-liquid separator 120 may be housed in the upper level 860. Further, the lower level 862 may be configured to define a separate space for the solids treatment arrangement 130 such that the solids treatment arrangement 130 may be housed in the lower level 362. According to various embodiments, the first casing structure 810a of the waste treatment device 800 may include a cover member 850 which is disposed in the upper level 860 and which removably cover the solid-liquid separator 120. FIG. 8B shows the cover member 850 being removed. As shown in FIG. 8B, according to various embodiments, the liquid discharge outlet 124 of the solid-liquid separator 120 may be connected to a first end of the piping 808. A second end of the piping 808 may be coupled to an inlet of the second casing structure 810b so as to direct the liquid separated by the solid-liquid separator 120 into the liquid treatment arrangement 140 embodied by the second casing structure 810b.

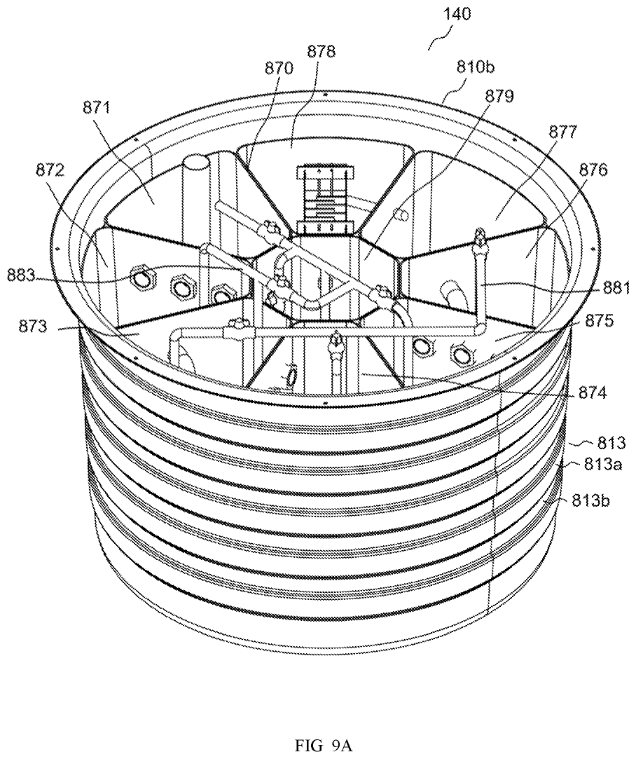

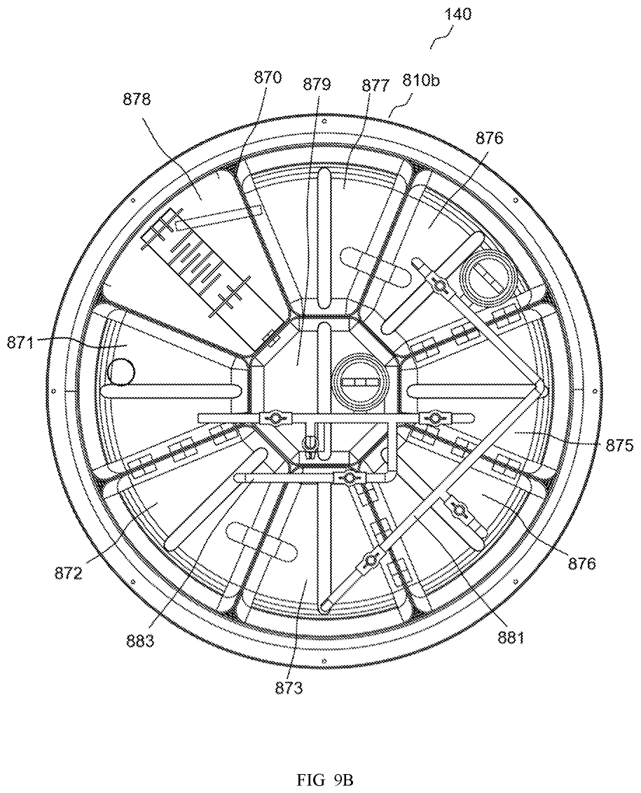

FIG. 9A and FIG. 9B shows a perspective view and a top view of the liquid treatment arrangement 140 of the waste treatment device 800 (which is embodied by the second casing structure 810b) with a cover 814 removed according to various embodiments. As shown, the second casing structure 810b may be of a cylindrical shape. According to various embodiments, the second casing structure 810b may include a cylindrical wall 813. The cylindrical wall 813 may include a plurality of raised ridges 813a and grooves 813b encircling the cylindrical wall 813 so as to form a series of corrugation around the cylindrical wall. Accordingly, the plurality of raised ridges 813a may be in the form of protruding rings around the cylindrical wall 813 and the plurality of grooves 813b may be in the form of recessed rings around the cylindrical wall 813. According to various embodiments, the protruding rings alternate with the recessed rings. According to various embodiments, the plurality of raised ridges 813a and grooves 813b may allow better adhesion and grip with the soil when the second casing structure 810b (i.e. the liquid treatment arrangement 140) is embedded in the ground.

As shown, according to various embodiments, the second casing structure 810b may be partitioned by partitioning walls 870 into nine chambers 871, 872, 873, 874, 875, 876, 877, 878, 879. The second casing structure 810b may be partitioned to have a middle chamber 879 configured to define a space in the centre of the cylindrically shape second casing structure 810b. Further, the second casing structure 810b may be partitioned to have eight side chambers 871, 872, 873, 874, 875, 876, 877, 878 distributed to surround the middle chamber 879. According to various embodiments, the eight side chambers 871, 872, 873, 874, 875, 876, 877, 878 may be assigned or allocated to the various processes of the liquid treatment arrangement 140, and the middle chamber 879 may be assigned or allocated to collect or store treated liquid.

For example, the first and second side chambers 871, 872 may be assigned or allocated to be the filtration chamber 241 of the liquid treatment arrangement 140. The first side chamber 871 may be an up-flow filter chamber whereby liquid separated by the solid-liquid separator 120 flows into the up-flow filter chamber (i.e. the first side chamber 871) upon entering the second casing structure 810b (i.e. the liquid treatment arrangement 140). The first side chamber 871 may include a plurality of plastic media for trapping residual solid waste or other solids constituents. The second side chamber 872 may be a first sedimentation chamber. Liquid from the first side chamber 871 may flow into the second side chamber 872 via a short upper pipeline. The second side chamber 872 may also include a plurality of plastic media for trapping residual solid waste or other solids constituents via a sedimentation process.

The third to sixth side chambers 873, 874, 875, 876 may be assigned or allocated for the various chambers (e.g. the anaerobic treatment chamber 243, the aerobic treatment chamber 245, the anoxic treatment chamber 247, and the holding chamber 249) of the biological treatment unit 242 of the liquid treatment arrangement 140. The third side chamber 873 may be the anaerobic treatment chamber 243 and may include a layer of a plurality of plastic media at the bottom and a layer of a plurality of spherical clay media at the bottom on top of the layer of the plurality of plastic media. The diameter of the spherical clay may be about 2 cm. The plurality of spherical clay media in the third side chamber 873 may decrease the value of chemical oxygen demand (COD) and nitrogen of the liquid. Liquid from the second side chamber 872 may flow into the third side chamber 873 via an inverted U pipeline. The fourth side chamber 874 may be the aerobic treatment chamber 245. The fourth side chamber 874 may include an aeration mechanism configured to supply air micro-bubbling so as to produce nitrogen dioxide and/or nitrate ion in the liquid. Liquid from the third side chamber 873 may flow into the fourth side chamber 874 via a short upper pipeline. The fifth side chamber 875 may be the anoxic treatment chamber 247. The fifth side chamber 875 may include a plurality of plastic media and spherical clay media, similar to the third side chamber 873 and the fourth side chamber 874. The diameter of the spherical clay in the fifth side chamber 875 may be about 1 cm. Under the no oxygen condition in the fifth side chamber 875, the nitrogen dioxide and/or nitrate ion in the liquid may be eliminated by biological nitrogen removal process to form Nitrogen. Liquid from the fourth side chamber 874 may flow into the fifth side chamber 875 via a short lower pipeline. The sixth side chamber 876 may be the holding chamber 249 (or the recirculation chamber). The sixth side chamber 876 may include a recirculation pump configured to circulate liquid back to the third side chamber 873 via a re-circulation pipeline 881 for re-treatment. Liquid from the fifth side chamber 875 may flow into the sixth side chamber 876 via a short upper pipeline.

The seventh side chamber 877 may be assigned or allocated for the sedimentation unit 244 of the liquid treatment arrangement 140. The seventh side chamber 877 may include a plurality of plastic media. Liquid from the sixth side chamber 876 may flow into the seventh side chamber 877 via an inverted U pipeline. The eight side chamber 878 may be assigned or allocated for the electrochemical unit 246. The electrochemical unit 246 may oxidize chloride ion in the liquid to chlorine for disinfection. Liquid from the seventh side chamber 877 may flow into eight side chamber 878 via a short upper pipeline. The middle chamber 879 which is the treated water chamber may receive liquid from the eight side chambers 878 via a short upper pipeline.

According to various embodiments, the liquid treatment arrangement 140 may include a backwash pipeline system 883. The backwash pipeline system 883 may connect the middle chamber 879 to the second side chamber 872, the third side chamber 873, and/or the fifth side chamber 875 for sending treated water back into the respective side chambers 872, 873, 875 for cleaning and/or washing the plastic media and/or spherical clay media. According to various embodiments, backwash cleaning may be performed at a frequency of once a year.

The following examples pertain to various embodiments.

Example 1 is a waste treatment device, including:
a solid-liquid separator configured to receive and separate waste into solids and liquid;
a solids treatment arrangement configured to receive the solids from the solid-liquid separator, wherein the solids treatment arrangement may include a disinfection unit having a heating mechanism configured to heat, without burning, the solids so as to disinfect the solids to convert the solids into pathogen-free-treated-solids; and
a liquid treatment arrangement which may be configured to receive the liquid from the solid-liquid separator and to treat the liquid so as to convert the liquid into pathogen-free-effluent,
wherein the solid-liquid separator comprises a curved-funnel-shaped inner separator surface configured to set the waste into spiral motion as the waste moves towards a spout of the curved-funnel-shaped inner separator surface,
wherein the solid-liquid separator comprises a frustoconically-shaped inner liquid guide surface, and wherein the spout of the curved-funnel-shaped inner separator surface and a narrower end of the frustoconically-shaped inner liquid guide surface are directed towards each other.

In Example 2, the subject matter of Example 1 may optionally include a single casing structure wherein the solid-liquid separator, the solids treatment arrangement and the liquid treatment arrangement may be integrated into the single casing structure.

In Example 3, the subject matter of Example 2 may optionally include that the single casing structure may include an upper level and a lower level, wherein the upper level may be configured to house the solid-liquid separator and the lower level may be configured to co-locate the solids treatment arrangement and the liquid treatment arrangement.

In Example 4, the subject matter of Example 3 may optionally include that the lower level may include at least one partitioning wall to divide the lower level into two or more chambers for demarcating separate areas for the solids treatment arrangement and the liquid treatment arrangement within the lower level.

In Example 5, the subject matter of any one of Examples 2 to 4 may optionally include that the single casing structure may be partitioned into three or more internal spaces for separately locating the solid-liquid separator, the solids treatment arrangement and the liquid treatment arrangement within the single casing structure.

In Example 6, the subject matter of any one of Examples 3 to 5 may optionally include that the single casing structure may include a suspended support frame which may separate the lower level and the upper level, and which may be configured to support the solid-liquid separator.

In Example 7, the subject matter of any one of Examples 2 to 6 may optionally include that the single casing structure may include a removable cover to enclose the solid-liquid separator.

In Example 8, the subject matter of Example 7 may optionally include that the single casing structure may include at least one other removable cover to cover over a portion of the lower level of the single casing structure.

In Example 9, the subject matter of any one of Examples 1 to 8 may optionally include that the solid-liquid separator may be configured to separate the waste into solids and liquid based on centrifugal force and gravity.

In Example 10, the subject matter of any one of Examples 1 to 9 may optionally include that the solid-liquid separator may further include a conduit portion, wherein the spout of the curved-funnel-shaped inner separator surface may be directly connected to a first end of the conduit portion and the narrower end of the frustoconically-shaped inner liquid guide surface may be directly connected to a second end of the conduit portion.

In Example 11, the subject matter of Example 10 may optionally include that a ratio of a length of the conduit portion to a height of the curved-funnel-shaped inner separator surface may be equal or less than 0.2.

In Example 12, the subject matter of any one of Examples 1 to 11 may optionally include that the heating mechanism of the disinfection unit of the solids treatment arrangement may be further configured to heat, without burning, the solids to remove moisture content from the solids so as to dry the solids.

In Example 13, the subject matter of any one of Examples 1 to 12 may optionally include that the heating mechanism of the disinfection unit of the solids treatment arrangement may be configured to heat an elongate housing of the disinfection unit so as to heat an internal space of the elongate housing to create a heated environment to apply thermal disinfection to the solids received inside the elongate housing from the solid-liquid separator.

In Example 14, the subject matter of any one of Examples 1 to 13 may optionally include that the heating mechanism may include one or more heating elements directly coupled to the elongate housing, and wherein the one or more heating elements may include a band heater, or a heater pad, or a heater plate, or a heating net, or a heater coil, or a heater wire, or a heater rod, or a heater fin, or any combination thereof, preferably the one or more heating elements may include the band heater.

In Example 15, the subject matter of any one of Examples 1 to 14 may optionally include that the disinfection unit may include a conveying mechanism configured to move the solids along and within the elongate housing, and wherein the conveying mechanism may include a screw conveyor mechanism, or a bucket conveyor mechanism, or a drag chain conveyor mechanism, or a belt conveyor mechanism, or a wire mesh conveyor mechanism, or a roller conveyor mechanism, or a spiral conveyor mechanism, preferably, the conveying mechanism may include the screw conveyor mechanism.

In Example 16, the subject matter of any one of Examples 1 to 15 may optionally include that the solids treatment arrangement may further include a collector unit which may be configured to receive the pathogen-free-treated-solids from the disinfection unit.

In Example 17, the subject matter of any one of Examples 1 to 16 may optionally include that the liquid treatment arrangement may include a biological treatment unit which may be configured to remove organic substances in the liquid.

In Example 18, the subject matter of Example 17 may optionally include that the biological treatment unit may include a filtration chamber, or an anaerobic treatment chamber, or an aerobic treatment chamber, or an anoxic treatment chamber, or any combination thereof.

In Example 19, the subject matter of Example 18 may optionally include that the biological treatment unit may include the filtration chamber, the anaerobic treatment chamber, the aerobic treatment chamber, and the anoxic treatment chamber, wherein the biological treatment unit may be configured to flow the liquid through the respective chambers in a sequence of the filtration chamber followed by the anaerobic treatment chamber followed by the aerobic treatment chamber and followed by the anoxic treatment chamber.

In Example 20, the subject matter of Example 18 or 19 may optionally include that the biological treatment unit may further include a recirculation pump configured to recirculate the liquid through the anaerobic treatment chamber, the aerobic treatment chamber, and the anoxic treatment chamber.

In Example 21, the subject matter of any one of Examples 1 to 20 may optionally include that the liquid treatment arrangement may include a sedimentation unit which may be configured to remove suspension particle in the liquid.

In Example 22, the subject matter of any one of Examples 1 to 21 may optionally include that the liquid treatment arrangement may include an electrochemical unit which may be configured to oxidize chloride-ion in the liquid to chlorine.

In Example 23, the subject matter of any one of Examples 17 to 22 may optionally include that the liquid treatment arrangement may further include a sedimentation unit and an electrochemical unit, and wherein the liquid treatment arrangement may be configured to flow the liquid through the respective units in a sequence of the biological treatment unit followed by the sedimentation unit and followed by the electrochemical unit.

In Example 24, the subject matter of any one of Examples 1 to 23 may optionally include that the liquid treatment arrangement may include an outlet configured to discharge the pathogen-free-effluent.

In Example 25, the subject matter of any one of Examples 22 to 24 may optionally include a fluid sensing sensor disposed at any point from the liquid discharge outlet 124 of the solid-liquid separator 120 to the liquid treatment arrangement 140.

In Example 26, the subject matter of Example 25 may optionally include that the disinfection unit of the solids treatment arrangement may further include one or more temperature sensors configured to measure a temperature of the internal space of the housing of the disinfection unit of the solids treatment arrangement.

In Example 27, the subject matter of Example 26 may optionally include a controller electrically coupled to the one or more temperature sensors of the disinfection unit of the solid treatment arrangement, the heating mechanism of the disinfection unit of the solid treatment arrangement, the conveying mechanism of the disinfection unit of the solid treatment arrangement, the electrochemical unit of the liquid treatment arrangement, the fluid sensing sensor, or any combination thereof.

In Example 28, the subject matter of Example 26 may optionally include a controller electrically coupled to the one or more temperature sensors of the disinfection unit of the solid treatment arrangement, the heating mechanism of the disinfection unit of the solid treatment arrangement, the conveying mechanism of the disinfection unit of the solid treatment arrangement, the electrochemical unit of the liquid treatment arrangement, and the fluid sensing sensor.

In Example 29, the subject matter of Example 28 may optionally include that the controller may be configured to activate the electrochemical unit upon detection of liquid flow from the solid-liquid separator to the liquid treatment arrangement by the fluid sensing sensor.

In Example 30, the subject matter of Example 29 may optionally include that the controller may be configured to count the number of times the fluid sensing sensor detects liquid flow from the solid-liquid separator to the liquid treatment arrangement.

In Example 31, the subject matter of Example 29 may optionally include that the controller may be configured to activate the conveying mechanism of the disinfection unit of the solids treatment arrangement based on a predetermined number of times the fluid sensing sensor detects liquid flow from the solid-liquid separator to the liquid treatment arrangement. The predetermined number of times may be between 10 to 20 times.

In Example 32, the subject matter of any one of Examples 28 to 31 may optionally include that the controller may be configured to activate the heating mechanism of the disinfection unit of the solids treatment arrangement based on a pre-set timing. The pre-set timing may be 12 midnight.

In Example 33, the subject matter of Example 32 may optionally include that the controller may be configured to control the heating mechanism of the disinfection unit of the solids treatment arrangement to maintain the temperature of the internal space of the elongate housing of the disinfection unit of the solids treatment arrangement at a predetermined disinfection temperature so as to maintain the heated environment within the elongate housing of the disinfection unit of the solids treatment arrangement. The predetermined disinfection temperature may be at least 70° C.

In Example 34, the subject matter of Example 33 may optionally include that the controller may be configured to control the conveying mechanism of the disinfection unit of the solids treatment arrangement to repeatedly move the solids in a first longitudinal direction and in a second longitudinal direction along the elongate housing of the disinfection unit of the solids treatment arrangement based on a predetermined sequence.

In Example 35, the subject matter of Example 33 or 34 may optionally include that the controller may be configured to activate the conveying mechanism of the disinfection unit of the solids treatment arrangement after the internal space of the elongate housing of the disinfection unit of the solids treatment arrangement is maintained at the predetermined temperature for a predetermined period of time.

In Example 36, the subject matter of Example 34 or 35 may optionally include that the controller may be configured to maintain the internal space of the housing of the disinfection unit of the solids treatment arrangement at the predetermined temperature while the conveying mechanism of the disinfection unit of the solids treatment arrangement is in operation.

Various embodiments have provided a waste treatment device that addresses the various issues identified earlier. For example, various embodiments have provided a single compact portable standalone waste treatment device that may be brought to rural places for direct treatment of waste or sewage collected in-situ before disposal or reusing the treated waste for agriculture. Various embodiments have also provided a waste treatment device that provides easy and fuss-free portable standalone all-in-one solution for direct treatment of waste or sewage in-situ in rural places without the need to fix up or install or connect multiple devices for processing the waste or sewage from toilet or lavatory or livestock farm to separate the solids and liquid, and for separately treating the solids and the liquid. Various embodiments have also provided a waste treatment device that may be configured to be installed directly to independent toilet or lavatory found in rural areas for direct independent treatment of waste or sewage. Various embodiments have also provided a compact portable all-in-one solution for separation of the solids and the liquid as well as treatment of both the solids and the liquid from the sewage of toilet or lavatory or livestock farm in one single device. Further, various embodiments have provided a waste treatment device that is capable of separating waste into solids and liquid, as well as treating the solids to disinfect and/or remove moisture from the solids so as to convert the solids into pathogen-free-treated-solids for direct agriculture application and treat the liquid to remove contaminants and organic substances so as to convert the liquid into pathogen-free-effluent.

While the invention has been particularly shown and described with reference to specific embodiments, it should be understood by those skilled in the art that various changes, modification, variation in form and detail may be made therein without departing from the scope of the invention as defined by the appended claims. The scope of the invention is thus indicated by the appended claims and all changes which come within the meaning and range of equivalency of the claims are therefore intended to be embraced.

1. A waste treatment device, comprising:
a solid-liquid separator which is configured to receive and separate waste into solids and liquid;
a solids treatment arrangement configured to receive the solids from the solid-liquid separator, wherein the solids treatment arrangement comprises a disinfection unit having a heating mechanism configured to heat, without burning, the solids so as to disinfect the solids to convert the solids into pathogen-free-treated-solids; and
a liquid treatment arrangement which is configured to receive the liquid from the solid-liquid separator and to treat the liquid so as to convert the liquid into pathogen-free-effluent,
wherein the solid-liquid separator comprises a curved-funnel-shaped inner separator surface configured to set the waste into spiral motion as the waste moves towards a spout of the curved-funnel-shaped inner separator surface,
wherein the solid-liquid separator comprises a frustoconically-shaped inner liquid guide surface, and wherein the spout of the curved-funnel-shaped inner separator surface and a narrower end of the frustoconically-shaped inner liquid guide surface are directed towards each other.

2. The waste treatment device as claimed in claim 1, further comprising a single casing structure wherein the solid-liquid separator, the solids treatment arrangement and the liquid treatment arrangement are integrated into the single casing structure.

3. The waste treatment device as claimed in claim 2, wherein the single casing structure comprises an upper level and a lower level, wherein the upper level is configured to house the solid-liquid separator and the lower level is configured to co-locate the solids treatment arrangement and the liquid treatment arrangement.

4. The waste treatment device as claimed in claim 3, wherein the lower level comprises at least one partitioning wall to divide the lower level into two or more chambers for demarcating separate areas for the solids treatment arrangement and the liquid treatment arrangement within the lower level.

5. The waste treatment device as claimed in claim 2, wherein the single casing structure is partitioned into three or more internal spaces for separately locating the solid-liquid separator, the solids treatment arrangement and the liquid treatment arrangement within the single casing structure.

6. The waste treatment device as claimed in claim 3, wherein the single casing structure comprises a suspended support frame which separates the lower level and the upper level, and which is configured to support the solid-liquid separator.

7. The waste treatment device as claimed in claim 2, wherein the single casing structure comprises a removable cover to enclose the solid-liquid separator.

8. The waste treatment device as claimed in claim 7, wherein the single casing structure comprises at least one other removable cover to cover over a portion of the lower level of the single casing structure.

9. The waste treatment device as claimed in claim 1, wherein the solid-liquid separator is configured to separate the waste into solids and liquid based on centrifugal force and gravity.

10. The waste treatment device as claimed in claim 1, wherein the solid-liquid separator further comprises a conduit portion, wherein the spout of the curved-funnel-shaped inner separator surface is directly connected to a first end of the conduit portion and the narrower end of the frustoconically-shaped inner liquid guide surface is directly connected to a second end of the conduit portion.

11. The waste treatment device as claimed in claim 10, wherein a ratio of a length of the conduit portion to a height of the curved-funnel-shaped inner separator surface is equal or less than 0.2.

12. The waste treatment device as claimed in claim 1, wherein the heating mechanism of the disinfection unit of the solids treatment arrangement is further configured to heat, without burning, the solids to remove moisture content from the solids so as to dry the solids.

13. The waste treatment device as claimed in claim 1, wherein the heating mechanism of the disinfection unit of the solids treatment arrangement is configured to heat an elongate housing of the disinfection unit so as to heat an internal space of the elongate housing to create a heated environment to apply thermal disinfection to the solids received inside the elongate housing from the solid-liquid separator.

14. The waste treatment device as claimed in claim 1, wherein the heating mechanism comprises one or more heating elements directly coupled to the elongate housing, and wherein the one or more heating elements comprises a band heater, or a heater pad, or a heater plate, or a heating net, or a heater coil, or a heater wire, or a heater rod, or a heater fin, or any combination thereof, preferably the one or more heating elements comprises the band heater.

15. The waste treatment device as claimed in claim 1, wherein the disinfection unit comprises a conveying mechanism configured to move the solids along and within the elongate housing, and wherein the conveying mechanism comprises a screw conveyor mechanism, or a bucket conveyor mechanism, or a drag chain conveyor mechanism, or a belt conveyor mechanism, or a wire mesh conveyor mechanism, or a roller conveyor mechanism, or a spiral conveyor mechanism, preferably, the conveying mechanism comprises the screw conveyor mechanism.

16. The waste treatment device as claimed in claim 1, wherein the solids treatment arrangement further comprises a collector unit which is configured to receive the pathogen-free-treated-solids from the disinfection unit.

17. The waste treatment device as claimed in claim 1, wherein the liquid treatment arrangement comprises a biological treatment unit which is configured to remove organic substances in the liquid.

18. The waste treatment device as claimed in claim 17, wherein the biological treatment unit comprises a filtration chamber, or an anaerobic treatment chamber, or an aerobic treatment chamber, or an anoxic treatment chamber, or any combination thereof.

19. The waste treatment device as claimed in claim 18, wherein the biological treatment unit comprises the filtration chamber, the anaerobic treatment chamber, the aerobic treatment chamber, and the anoxic treatment chamber, wherein the biological treatment unit is configured to flow the liquid through the respective chambers in a sequence of the filtration chamber followed by the anaerobic treatment chamber followed by the aerobic treatment chamber and followed by the anoxic treatment chamber.

20. The waste treatment device as claimed in claim 18, wherein the biological treatment unit further comprises a recirculation pump configured to recirculate the liquid through the anaerobic treatment chamber, the aerobic treatment chamber, and the anoxic treatment chamber.

21. The waste treatment device as claimed in claim 1, wherein the liquid treatment arrangement comprises a sedimentation unit which is configured to remove suspension particle in the liquid.

22. The waste treatment device as claimed in claim 1, wherein the liquid treatment arrangement comprises an electrochemical unit which is configured to oxidize chloride-ion in the liquid to chlorine.

23. The waste treatment device as claimed in claim 17, wherein the liquid treatment arrangement further comprises a sedimentation unit and an electrochemical unit, and wherein the liquid treatment arrangement is configured to flow the liquid through the respective units in a sequence of the biological treatment unit followed by the sedimentation unit and followed by the electrochemical unit.

24. The waste treatment device as claimed in claim 1, wherein the liquid treatment arrangement comprises an outlet configured to discharge the pathogen-free-effluent.

25. The waste treatment device as claimed in claim 22, further comprising a fluid sensing sensor disposed at any point from the liquid discharge outlet of the solid-liquid separator to the liquid treatment arrangement.

26. The waste treatment device as claimed in claim 25, wherein the disinfection unit of the solids treatment arrangement further comprises one or more temperature sensors configured to measure a temperature of the internal space of the housing of the disinfection unit of the solids treatment arrangement.

27. The waste treatment device as claimed in claim 26, further comprising a controller electrically coupled to the one or more temperature sensors of the disinfection unit of the solid treatment arrangement, the heating mechanism of the disinfection unit of the solid treatment arrangement, the conveying mechanism of the disinfection unit of the solid treatment arrangement, the electrochemical unit of the liquid treatment arrangement, the fluid sensing sensor, or any combination thereof.

28. The waste treatment device as claimed in claim 26, further comprising a controller electrically coupled to the one or more temperature sensors of the disinfection unit of the solid treatment arrangement, the heating mechanism of the disinfection unit of the solid treatment arrangement, the conveying mechanism of the disinfection unit of the solid treatment arrangement, the electrochemical unit of the liquid treatment arrangement, and the fluid sensing sensor.

29. The waste treatment device as claimed in claim 28, wherein the controller is configured to activate the electrochemical unit upon detection of liquid flow from the solid-liquid separator to the liquid treatment arrangement by the fluid sensing sensor.

30. The waste treatment device as claimed in claim 29, wherein the controller is configured to count the number of times the fluid sensing sensor detects liquid flow from the solid-liquid separator to the liquid treatment arrangement.

31. The waste treatment device as claimed in claim 30, wherein the controller is configured to activate the conveying mechanism of the disinfection unit of the solids treatment arrangement based on a predetermined number of times the fluid sensing sensor detects liquid flow from the solid-liquid separator to the liquid treatment arrangement.

32. The waste treatment device as claimed in claim 28, wherein the controller is configured to activate the heating mechanism of the disinfection unit of the solids treatment arrangement based on a pre-set timing.

33. The waste treatment device as claimed in claim 32, wherein the controller is configured to control the heating mechanism of the disinfection unit of the solids treatment arrangement to maintain the temperature of the internal space of the elongate housing of the disinfection unit of the solids treatment arrangement at a predetermined disinfection temperature so as to maintain the heated environment within the elongate housing of the disinfection unit of the solids treatment arrangement.

34. The waste treatment device as claimed in claim 33, wherein the controller is configured to control the conveying mechanism of the disinfection unit of the solids treatment arrangement to repeatedly move the solids in a first longitudinal direction and in a second longitudinal direction along the elongate housing of the disinfection unit of the solids treatment arrangement based on a predetermined sequence.

35. The waste treatment device as claimed in claim 33, wherein the controller is configured to activate the conveying mechanism of the disinfection unit of the solids treatment arrangement after the internal space of the elongate housing of the disinfection unit of the solids treatment arrangement is maintained at the predetermined temperature for a predetermined period of time.

36. The waste treatment device as claimed in claim 34, wherein the controller is configured to maintain the internal space of the housing of the disinfection unit of the solids treatment arrangement at the predetermined temperature while the conveying mechanism of the disinfection unit of the solids treatment arrangement is in operation.
